(19) United States
(12) Patent Application Publication
(10) Pub. No.: US 2005/0288304 A1
(43) Pub. Date: Dec. 29, 2005

(54) Tetrahydronaphthylpiperazines as 5HT1B antagonists, inverse agonists and partial agonists

(75) Inventors: Michael A. Brodney, East Lyme, CT (US); Karen J. Coffman, Pawcatuck, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/135,215

(22) Filed: May 23, 2005

Related U.S. Application Data
(60) Provisional application No. 60/573,557, filed on May 21, 2004.

(51) Int. Cl. A61K 31/495; C07D 241/04

(52) U.S. Cl. 514/255.03; 544/392

(57) ABSTRACT

The present invention relates to novel tetrahydronaphthylpiperazines derivatives, that are compounds of the formula I 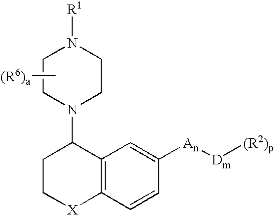
wherein R¹, R² and R⁶ are as defined herein, X is CH₂ or O, A is a group of the formula G¹, G², G^{2a}, G³, G⁴, G⁵ or G⁶ depicted below, 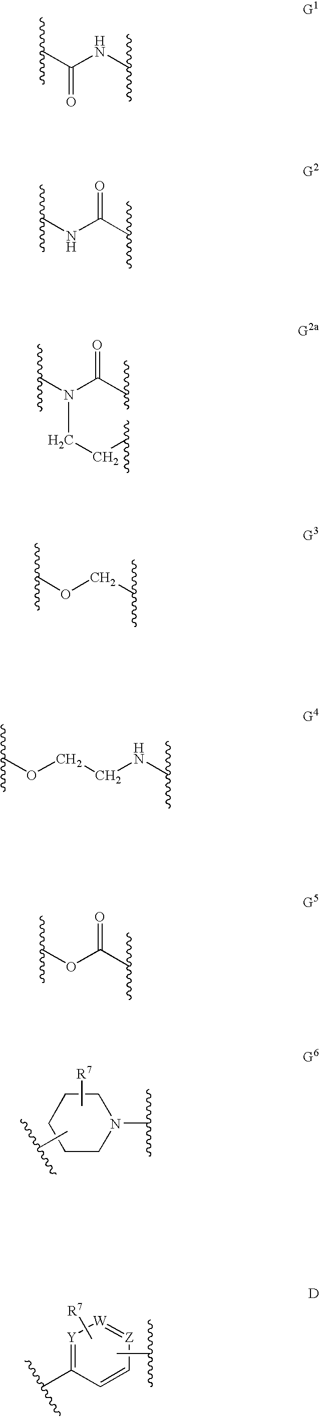
and D is a group of the formula D, wherein Y, W and Z are C or N and wherein R⁷ is as defined herein and their salts and compositions which include selective antagonists, inverse agonists and partial agonists of serotonin 1 (5-HT₁) receptors. Compounds of the invention are useful in treating or preventing depression, anxiety, obsessive compulsive disorder (OCD) and other disorders for which a 5-HT₁ agonist or antagonist is indicated.

### BACKGROUND OF THE INVENTION

The present invention relates to novel tetrahydronaphthylpiperazines derivatives, to intermediates for their preparation, to pharmaceutical compositions containing them and to their medicinal use. The compounds of the present invention include selective antagonists, inverse agonists and partial agonists of serotonin 1 (5-HT₁) receptors, specifically 5-HT_{1B} (formerly classified 5-HT_{1D}) receptors or combination of 5-HT_{1B} and 5-HT_{1A} receptors. They are useful in treating or preventing depression, anxiety, obsessive compulsive disorder (OCD) and other disorders for which a 5-HT₁ agonist or antagonist is indicated.

European Patent Publication 434,561, published on Jun. 26, 1991, refers to 7-alkyl alkoxy, and hydroxy substituted-1-(4-substituted-1-piperazinyl)naphthalenes. The compounds are referred to as 5-HT, agonists and antagonists useful for the treatment of migraine, depression, anxiety, schizophrenia, stress and pain.

European Patent Publication 343,050, published on Nov. 23, 1989, refers to 7-unsubstituted, halogenated, and methoxy substituted-1-(4-substituted-1-piperazinyl) naphthalenes as useful 5-HT_{1A} ligand therapeutics.

PCT publication WO 94/21619, published Sep. 29, 1994, refers to naphthalene derivatives as 5-HT, agonists and antagonists.

PCT publication WO 96/00720, published Jan. 11, 1996, refers to naphthyl ethers as useful 5-HT, agonists and antagonists.

European Patent Publication 701,819, published Mar. 20, 1996, refers to the use of 5-HT, agonists and antagonists in combination with a 5-HT re-uptake inhibitor.

Glennon et al., refers to 7-methoxy-1-(1-piperazinyl)-naphthalene as a useful 5-HT₁ ligand in their article “5-HT_{1D} Serotonin Receptors”, Clinical Drug Res. Dev., 22, 25-36 (1991).

Glennon's article “Serotonin Receptors: Clinical Implications”, Neuroscience and Behavioral Reviews, 14, 3547 (1990), refers to the pharmacological effects associated with serotonin receptors including appetite suppression, thermoregulation, cardiovascular/hypotensive effects, sleep, psychosis, anxiety, depression, nausea, emesis, Alzheimer's disease, Parkinson's disease and Huntington's disease.

World Patent Application WO 95/31988, published Nov. 30, 1995, refers to the use of a 5-HT_{1D} antagonist in combination with a 5-HT_{1A} antagonist to treat CNS disorders such as depression, generalized anxiety, panic disorder, agoraphobia, social phobias, obsessive-compulsive disorder, post-traumatic stress disorder, memory disorders, anorexia nervosa and bulimia nervosa, Parkinson's disease, tardive dyskinesias, endocrine disorders such as hyperprolactinaemia, vasospasm (particularly in the cerebral vasculature) and hypertension, disorders of the gastrointestinal tract where changes in motility and secretion are involved, as well as sexual dysfunction.

G. Maura et al., J. Neurochem, 66 (1), 203-209 (1996), have stated that administration of agonists selective for 5-HT_{1A} receptors or for both 5-HT_{1A} and 5-HT_{1D} receptors might represent a great improvement in the treatment of human cerebellar ataxias, a multifaceted syndrome for which no established therapy is available.

European Patent Publication 666,261, published Aug. 9, 1995 refers to thiazine and thiomorpholine derivatives which are claimed to be useful for the treatment of cataracts.

PCT International Publications WO99/05134, published Feb. 4, 1999, and WO97/34883, published Sep. 25, 1997, refer to substituted 1,2,3,4-tetrahydronaphthalenes asserted to be useful for treating 5-HT-mediated disorders.

### SUMMARY OF THE INVENTION

The present invention relates to tetrahydronaphthylpiperazines of the formula 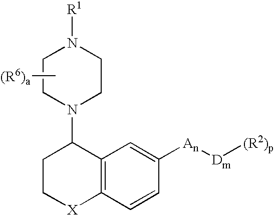
wherein
X is CH₂ or O;
n is zero or one;
m is zero or one;
p is zero or one;
R¹ is hydrogen, (C₁-C₆)alkyl, (C₁-C₄)alkyl-aryl wherein said aryl moiety is phenyl or naphthyl, wherein said aryl moiety may optionally be substituted with one or more substituents independently selected from (C₁-C₆)alkyl, (C₁-C₆)alkoxy, trifluoromethyl, cyano and halo;
A is absent or a group of the formula G¹, G², G^{2a}, G³, G⁴, G⁵, and G⁶ depicted below, 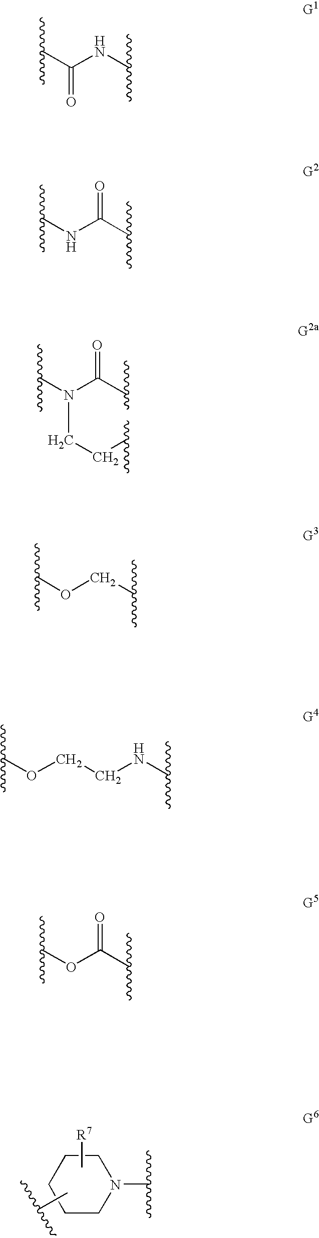
   wherein the C═O and CH₂ of G^{2a} bind to two adjacent carbon atoms of the D moiety so that G^{2a} forms a six membered ring including two adjacent carbon atoms of the D moiety;
wherein if n is zero and m is one, the tetrahydronaphthyl moiety bonds to D, and if both n and m are zero and p is one the tetrahydronaphthyl moiety bonds to R², and if n is one, m is zero and p is one A bonds to R²;
D is a group of the formula depicted below, 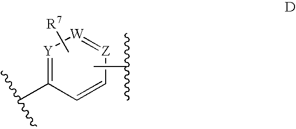
   wherein W, Y and Z are independently C or N;
wherein R⁷ is H or optionally one to four substituents independently selected from chloro, fluoro, bromo, iodo, (C₁-C₈)alkyl, (C₁-C₈)perfluoroalkyl, wherein said alkyl or perfluoroalkyl is branched or linear, (C₁-C₈)hydroxyalkyl-, —CH₂NR⁸R⁹, wherein R⁸ and R⁹ are independently H or (C₁-C₈)alkyl-, (C₁-C₈)alkoxy, (C₄-C₈)cycloalkyloxy, (C₄-C₈)cycloalkenyloxy, (C₁-C₈)alkoxy-(C₁-C₈)alkyl-, or R⁷ is a 5 to 7 membered non-aromatic heterocyclic ring having in addition to carbon atoms one to three heteroatoms independently selected from nitrogen, oxygen or sulfur atom or any combination thereof with the proviso that said ring cannot contain two adjacent oxygen atoms or two adjacent sulfur atoms; or,
R⁷ is —CONR⁴R⁵ wherein R⁴ and R⁵ are independently selected from (C₁-C₈)alkyl, (C₁-C₈)alkoxy benzyl, or R⁴ and R⁵ together with the nitrogen to which they are attached form a 5 to 7 membered heteroalkyl ring that may contain from zero to three heteroatoms selected from nitrogen, sulfur and oxygen in addition to the nitrogen of the —CONR⁴R⁵ group, wherein when any of said heteroatoms is nitrogen it may be optionally substituted with (C₁-C₈)alkyl or benzyl, with the proviso that said ring cannot contain two adjacent oxygen atoms or two adjacent sulfur atoms;
wherein R² is H, (C₁-C₈)alkyl, aryl, heteroaryl, aryl(C₁-C₆)alkyl-, aryl(C₁-C₆)alkyl-O—, aryl-(C═O)—, heteroaryl(C₁-C₆)alkyl-, hetereoaryl(C₁-C₆)O—, heteroaryl-(C═O)— wherein aryl is phenyl or naphthyl, and heteroaryl is a 5 to 7 membered aromatic ring containing from one to four heteroatoms in the ring selected from oxygen, nitrogen and sulfur, with the proviso that said ring cannot contain two adjacent oxygen atoms or two adjacent sulfur atoms and wherein the foregoing phenyl, naphthyl, and heteroaryl rings may be optionally substituted with one to three substituents independently selected from chloro, fluoro, bromo, iodo, (C₁-C₈)alkyl, (C₁-C₈)perfluoroalkyl, wherein said alkyl or perfluoroalkyl is branched or linear, (C₁-C₈)hydroxyalkyl-, (C₁-C₈)alkoxy, (C₁-C₈)alkoxy-(C₁-C₈)alkyl-, aryl, heteroaryl, aryl(C₁-C₆)alkyl-, aryl(C₁-C₆)alkyl-O—, aryl-(C═O)—, heteroaryl(C₁-C₆)alkyl-, hetereoaryl(C₁-C₆)O—, heteroaryl-(C═O)— wherein aryl is phenyl, naphthyl or 1,2,3,4-tetrahydro-naphthalenyl, and heteroaryl is a 5 to 7 membered aromatic ring containing from one to four heteroatoms in the ring selected from oxygen, nitrogen and sulfur, with the proviso that said ring cannot contain two adjacent oxygen atoms or two adjacent sulfur atoms;
R⁶ is (C₁-C₈)alkyl, wherein said alkyl is branched or linear; and,
a is 1 to 8, preferably one, two or three;
or, a pharmaceutically acceptable salt thereof.

The invention also relates to a compound according to formula I wherein R² is aryl, heteroaryl, aryl(C₁-C₆)alkyl-, aryl(C₁-C₆)alkyl-O—, aryl-(C═O)—, heteroaryl(C₁-C₆)alkyl-, hetereoaryl(C₁-C₆)O—, heteroaryl-(C═O)— wherein aryl is phenyl, naphthyl or 1,2,3,4-tetrahydro-naphthalenyl, and heteroaryl is selected from pyridyl, pyrrolyl, pyrimidyl, pyrazolyl, imidazolyl, triazolyl, tetrazolyl, oxazolyl, isoxazolyl, oxadiazolyl, thienyl, thiazolyl and isothiazoly and wherein the foregoing phenyl, naphthyl, and heteroaryl rings may be optionally substituted with one to three substituents independently selected from chloro, fluoro, bromo, iodo, (C₁-C₈)alkyl, (C₁-C₈)perfluoroalkyl, wherein said alkyl or perfluoroalkyl is branched or linear, (C₁-C₈)hydroxyalkyl-, (C₁-C₈)alkoxy, (C₁-C₈)alkoxy-(C₁-C₈)alkyl-, aryl, heteroaryl, aryl(C₁-C₆)alkyl-, aryl(C₁-C₆)alkyl-O—, aryl-(C═O)—, heteroaryl(C₁-C₆)alkyl-, hetereoaryl(C₁-C₆)O—, heteroaryl-(C═O)— wherein aryl is phenyl, naphthyl or and heteroaryl is selected from pyridyl, pyrrolyl, pyrimidyl, pyrazolyl, imidazolyl, triazolyl, tetrazolyl, oxazolyl, isoxazolyl, oxadiazolyl, thienyl, thiazolyl and isothiazoly.

The invention also relates to a compound according to formula I wherein R⁷ is one to three substituents independently selected from the group consisting of phenyl, naphthyl, tetrahydropyranyl, morpholinyl, azetidinyl, pyrrolidinyl, piperidyl, piperazinyl, morpholinyl, thiomorpholinyl, hexahydroazepinyl, diazepinyl, oxazepinyl, thiazepinyl, oxadiazepinyl, thiadiazepinyl or triazepinyl, oxetanyl, tetrahydrofuranyl and wherein each said substituent may be independently substituted with from zero to three substituents independently selected from (C₁-C₆)alkyl chloro, fluoro, bromo, iodo, (C₁-C₈)alkyl, (C₁-C₈)perfluoroalkyl, wherein said alkyl or perfluoroalkyl is branched or linear, (C₁-C₈)hydroxyalkyl-, (C₁-C₈)alkoxy, (C₁-C₈)alkoxy-(C₁-C₈)alkyl-, aryl(C₁-C₆)alkyl.

The invention also relates to a compound according to formula I wherein R⁷ is —CONR⁴R⁵ wherein R⁴ and R⁵ together with the nitrogen to which they are attached form a heteroalkyl ring selected from piperidine, N-(C₁-C₆)alkylpiperazine and morpholine.

The invention also relates to a compound according to formula I wherein X is CH₂.

The invention also relates to a compound according to formula I wherein X is O.

The invention also relates to a compound according to formula I wherein W, Y and Z are carbon.

The invention also relates to a compound according to formula I wherein one of W, Y or Z is nitrogen.

The invention also relates to a compound according to formula I wherein n is zero.

The invention also relates to a compound according to formula I wherein n is one.

The invention also relates to a compound according to formula I wherein m is zero.

The invention also relates to a compound according to formula I wherein m is one.

The invention also relates to a compound according to formula I wherein p is zero.

The invention also relates to a compound according to formula I wherein p is one.

The invention also relates to a compound according to formula I wherein n, m and p are one.

The invention also relates to a compound according to formula I wherein n is zero, m is one and p is one.

The invention also relates to a compound according to formula I wherein n is zero, m is zero and p is one.

The invention also relates to a compound according to formula I wherein n is one, m is zero and p is one.

The invention also relates to a compound according to formula I wherein n is one, m is one and p is zero.

The invention also relates to a compound according to formula I wherein R¹ is selected from hydrogen, (C₁-C₆)alkyl, (C₁-C₄)alkyl-aryl wherein said aryl moiety is phenyl or naphthyl.

The invention also relates to a compound according to formula I wherein R¹ is selected from hydrogen, methyl, ethyl and benzyl.

Specific examples of the compounds of the present invention are as follows:
N-{8-[(2-Dimethylamino-ethyl)-ethyl-amino]-5,6,7,8-tetrahydro-naphthalen-2-yl}-4-trifluoromethyl-benzamide;
N-{8-[(2-Dimethylamino-ethyl)ethyl-amino]-5,6,7,8-tetrahydro-naphthalen-2-yl}-4-fluoro-benzamide;
4-tert-Butyl-N-{8-[(2-dimethylamino-ethyl)-ethyl-amino]-5,6,7,8-tetrahydro-naphthalen-2-yl}-benzamide;
1-[7-(4-Benzyl-phenyl) 1,2,3,4-tetrahydro-naphthalen-1-yl]-4-methyl-piperazine;
1-[7-(4-Benzyloxy-phenyl)-1,2,3,4-tetrahydro-naphthalen-1-yl]-4-methyl-piperazine;
1-Methyl-4-(7-phenyl-1,2,3,4-tetrahydro-naphthalen-1-yl)-piperazine;
1-[7-(4-Fluoro-phenyl)-1,2,3,4-tetrahydro-naphthalen-1-yl]-4-methyl-piperazine;
1-[7-(3,5-Dichloro-phenyl)-1,2,3,4-tetrahydro-naphthalen-1-yl]-4-methyl-piperazine;
1-[7-(2-Methoxy-phenyl)-1,2,3,4-tetrahydro-naphthalen-1-yl]-4-methyl-piperazine;
1-Methyl-4-[7-(4-trifluoromethyl-phenyl)-1,2,3,4-tetrahydro-naphthalen-1-yl]-piperazine;
1-[7-(3,4-Dimethoxy-phenyl)-1,2,3,4-tetrahydro-naphthalen-1-yl]-4-methyl-piperazine;
1-(7-Biphenyl-4-yl-1,2,3,4-tetrahydro-naphthalen-1-yl)-4-methyl-piperazine;
1-[7-(2,3-Dihydro-benzo[1,4]dioxin-6-yl)-1,2,3,4-tetrahydro-naphthalen-1-yl]-4-methyl-piperazine;
8-(4-Methyl-piperazin-1-yl)-5,6,7,8-tetrahydro-naphthalene-2-carboxylic acid [1-(4-methoxy-phenyl)ethyl]-amide;
8-(4-Methyl-piperazin-1-yl)-5,6,7,8-tetrahydro-naphthalene-2-carboxylic acid 4-tert-butyl-benzylamide;
8-(4-Methyl-piperazin-1-yl)-5,6,7,8-tetrahydro-naphthalene-2-carboxylic acid 4-trifluoromethyl-benzylamide;
8-(4-Methyl-piperazin-1-yl)-5,6,7,8-tetrahydro-naphthalene-2-carboxylic acid (1,2,3,4-tetrahydro-naphthalen-1-yl)-amide;
8-(4-Methyl-piperazin-1-yl)-5,6,7,8-tetrahydro-naphthalene-2-carboxylic acid [2-(3-trifluoromethyl-phenyl)-ethyl]-amide;
8-(4-Methyl-piperazin-1-yl)-5,6,7,8-tetrahydro-naphthalene-2-carboxylic acid 4-chloro-benzylamide;
8-(4-Methyl-piperazin-1-yl)-5,6,7,8-tetrahydro-naphthalene-2-carboxylic acid 4-fluoro-benzylamide;
8-(4-Methyl-piperazin-1-yl)-5,6,7,8-tetrahydro-naphthalene-2-carboxylic acid (furan-2-ylmethyl)-amide;
8-(4-Methyl-piperazin-1-yl)-5,6,7,8-tetrahydro-naphthalene-2-carboxylic acid 2-chloro-benzylamide;
8-(4-Methyl-piperazin-1-yl)-5,6,7,8-tetrahydro-naphthalene-2-carboxylic acid 3-trifluoromethyl-benzylamide;
8-(4-Methyl-piperazin-1-yl)-5,6,7,8-tetrahydro-naphthalene-2-carboxylic acid 3-fluoro-benzylamide;
8-(4-Methyl-piperazin-1-yl)-5,6,7,8-tetrahydro-naphthalene-2-carboxylic acid 2-methyl-benzylamide;
8-(4-Methyl-piperazin-1-yl)-5,6,7,8-tetrahydro-naphthalene-2-carboxylic acid 2-methoxy-benzylamide;
8-(4-Methyl-piperazin-1-yl)-5,6,7,8-tetrahydro-naphthalene-2-carboxylic acid 3-methoxy-benzylamide;
8-(4-Methyl-piperazin-1-yl)-5,6,7,8-tetrahydro-naphthalene-2-carboxylic acid (2,5-dimethyl-2H-pyrazol-3-yl)-amide;
8-(4-Methyl-piperazin-1-yl)-5,6,7,8-tetrahydro-naphthalene-2-carboxylic acid (5-tert-butyl-2-methyl-2H-pyrazol-3-yl)-amide;
8-(4-Methyl-piperazin-1-yl)-5,6,7,8-tetrahydro-naphthalene-2-carboxylic acid [1-(4-chloro-phenylyethyl]-amide;
8-(4-Methyl-piperazin-1-yl)-5,6,7,8-tetrahydro-naphthalene-2-carboxylic acid 3-chloro-benzylamide;
8-(4-Methyl-piperazin-1-yl)-5,6,7,8-tetrahydro-naphthalene-2-carboxylic acid (pyridin-2-ylmethyl)-amide;
8-(4-Methyl-piperazin-1-yl)-5,6,7,8-tetrahydro-naphthalene-2-carboxylic acid (4-chloro-phenyl)amide;
8-(4-Methyl-piperazin-1-yl)-5,6,7,8-tetrahydro-naphthalene-2-carboxylic acid (5-methyl-[1,3,4]thiadiazol-2-yl)-amide;
8-(4-Methyl-piperazin-1-yl)-5,6,7,8-tetrahydro-naphthalene-2-carboxylic acid 3,4-difluoro-benzylamide;
8-(4-Methyl-piperazin-1-yl)-5,6,7,8-tetrahydro-naphthalene-2-carboxylic acid 4-methoxy-benzylamide;
8-(4-Methyl-piperazin-1-yl)-5,6,7,8-tetrahydro-naphthalene-2-carboxylic acid pyridin-2-ylamide;
1-Methyl-4-(7-o-tolyl-1,2,3,4-tetrahydro-naphthalen-1-yl)-piperazine;
1-[7-(3,4-Dichloro-phenyl)-1,2,3,4-tetrahydro-naphthalen-1-yl]-4-methyl-piperazine;
1-[7-(3-Methoxy-phenyl)-1,2,3,4-tetrahydro-naphthalen-1-yl]-4-methyl-piperazine;
4-tert-Butyl-N-[8-(4-methyl-piperazin-1-yl)-5,6,7,8-tetrahydro-naphthalen-2-yl]-benzamide;
2-Methoxy-N-[8-(4-methyl-piperazin-1-yl)-5,6,7,8-tetrahydro-naphthalen-2-yl]-4-morpholin-4-yl-benzamide;
4-Isopropoxy-N-[8-(4-methyl-piperazin-1-yl)-5,6,7,8-tetrahydro-naphthalen-2-yl]-benzamide;
4-Benzyloxy-N-[8-(4-methyl-piperazin-1-yl)-5,6,7,8-tetrahydro-naphthalen-2-yl]-benzamide;
Benzo[1,3]dioxole-5-carboxylic acid [8-(4-methyl-piperazin-1-yl)-5,6,7,8-tetrahydro-naphthalen-2-yl]-amide;
4-(Cyclohex-1-enyloxyyN-[8-(4-methyl-piperazin-1-yl)-5,6,7,8-tetrahydro-naphthalen-2-yl]-benzamide;
N-{8-[(2-Dimethylamino-ethyl)methyl-amino]-5,6,7,8-tetrahydro-naphthalen-2-yl}2-fluoro-4-morpholin-4-yl-benzamide;
N-[8-(4-Methyl-piperazin-1-yl)-5,6,7,8-tetrahydro-naphthalen-2-yl]-4-trifluoromethoxy-benzamide;
1-Methyl-4-(7-pyridin-4-yl-1,2,3,4-tetrahydro-naphthalen-1-yl)-piperazine;
1-Methyl-4-[7-(4-methyl-pyridin-3-yl)-1,2,3,4-tetrahydro-naphthalen-1-yl]-piperazine;
1-Methyl-4-[7-(6-methyl-pyridin-3-yl)-1,2,3,4-tetrahydro-naphthalen-1-yl]-piperazine;
1-[7-(6-Methoxy-pyridin-3-yl)-1,2,3,4-tetrahydro-naphthalen-1-yl]-4-methyl-piperazine;
1-Methyl-4-(7-pyridin-2-yl-1,2,3,4-tetrahydro-naphthalen-1-yl)-piperazine;
4-{5-[8-(4-Methyl-piperazin-1-yl)-5,6,7,8-tetrahydro-naphthalen-2-yl]-pyridin-2-yl}-morpholine;
(+)-1-Methyl-4-[7-(4-methyl-pyridin-3-yl)-1,2,3,4-tetrahydro-naphthalen-1-yl]-piperazine;
(−)-1-Methyl-4-[7-(4-methyl-pyridin-3-yl)-1,2,3,4-tetrahydro-naphthalen-1-yl]-piperazine;
1-Methyl-4-[6-(4-methyl-pyridin-3-yl)-chroman-4-yl]-piperazine;
1-Methyl-4-(6-pyridin-4-yl-chroman-4-yl)-piperazine;
4-{5-[4-(4-Methyl-piperazin-1-yl)-chroman-6-yl]-pyridin-3-yl}-morpholine;
1-[4-(4-Methyl-piperazin-1-yl)-chroman-6-yl]-1H-pyrrolo[2,3-b]pyridine;
1-Methyl-4-[6-(4-methyl-pyridin-3-yl)-chroman-4-yl]-piperazine;
1-Methyl-4-[7-(4-trifluoromethyl-benzyloxy)-1,2,3,4-tetrahydro-naphthalen-1-yl]-piperazine;
1-[7-(4-tert-Butyl-benzyloxy)-1,2,3,4-tetrahydro-naphthalen-1-yl]-4-methyl-piperazine;
6-Morpholin-4-yl-nicotinic acid 8-(4-methyl-piperazin-1-yl)-5,6,7,8-tetrahydro-naphthalen-2-yl ester;
N-[8-(4-Methyl-piperazin-1-yl)-5,6,7,8-tetrahydro-naphthalen-2-yl]-6-morpholin-4-yl-nicotinamide;
{4-[8-(4-Methyl-piperazin-1-yl)-5,6,7,8-tetrahydro-naphthalen-2-ylcarbamoyl]-benzyl}-carbamic acid tert-butyl ester;
N-[8-(4-Methyl-piperazin-1-yl)-5,6,7,8-tetrahydro-naphthalen-2-yl]-4-trifluoromethyl-benzamide;
N-[8-(4-Methyl-piperazin-1-yl)-5,6,7,8-tetrahydro-naphthalen-2-yl]-4-trifluoromethyl-benzamide;
2-[8-(4-Methyl-piperazin-1-yl)-5,6,7,8-tetrahydro-naphthalen-2-yl]-6-morpholin-4-yl-3,4-dihydro-2H-isoquinolin-1-one;
1-Methyl-4-(7-piperidin-4-yl-1,2,3,4-tetrahydro-naphthalen-1-yl)-piperazine;
1-Methyl-4-(7-piperidin-3-yl-1,2,3,4-tetrahydro-naphthalen-1-yl)-piperazine;
1-Methyl-4-[7-(1-methyl-piperidin-4-yl)-1,2,3,4-tetrahydro-naphthalen-1-yl]-piperazine;
(5-Fluoro-pyrimidin-2-yl)-{2-[8-(4-methyl-piperazin-1-yl)-5,6,7,8-tetrahydro-naphthalen-2-yloxy]-ethyl}amine.
N-{2-[8-(4-Methyl-piperazin-1-yl)-5,6,7,8-tetrahydro-naphthalen-2-yloxy]-ethyl}4-trifluoromethyl-benzamide;
N-{2-[8-(4-Methyl-piperazin-1-yl)-5,6,7,8-tetrahydro-naphthalen-2-yloxy]-ethyl}-4-trifluoromethyl-benzamide;
{4-[8-(4-Methyl-piperazin-1-yl)-5,6,7,8-tetrahydro-naphthalen-2-yl]-piperidin-1-yl}(4-trifluoromethyl-phenyl)-methanone;
{3-[8-(4-Methyl-piperazin-1-yl)-5,6,7,8-tetrahydro-naphthalen-2-yl]-piperidin-1-yl}(4-trifluoromethyl-phenyl)methanone;
4-Aminomethyl-N-[8-(4-methyl-piperazin-1-yl)-5,6,7,8-tetrahydro-naphthalen-2-yl]-benzamide;
(+)-1-Methyl-4-(7-pyridin-4-yl-1,2,3,4-tetrahydro-naphthalen-1-yl)-piperazine;
(−)-1-Methyl-4-(7-pyridin-4-yl-1,2,3,4-tetrahydro-naphthalen-1-yl)piperazine;
4-(1-Hydroxy-1-methyl-ethyl)-N-[8-(4-methyl-piperazin-1-yl)-5,6,7,8-tetrahydro-naphthalen-2-yl]-benzamide;
3-[8-(4-Methyl-piperazin-1-yl)-5,6,7,8-tetrahydro-naphthalen-2-yl]-6′-morpholin-4-yl-3,4,5,6-tetrahydro-2H-[1,2′]bipyridinyl;
   (+)- and (−)-enantiomers thereof; and, pharmaceutically acceptable salts thereof.

Unless otherwise indicated, the term “halo”, as used herein, includes fluoro, chloro, bromo and iodo.

Unless otherwise indicated, the term “alkyl”, as used herein, includes straight or branched alkyl.

Unless otherwise indicated the term “cycloalkyl” as used herein includes moieties derived from cyclic hydrocarbons which have a linkage from a ring carbon to another group and includes cyclic hydrocarbon moieties substituted with straight or branched alkyl moieties.

The term “alkoxy”, as used herein, means “alkyl-O—”, wherein “alkyl” is defined as above.

The term “cycloalkyl-O—” as used herein means “cycloalkyl” as defined above in which the cycloalkyl moiety is linked by a single bond to an oxygen atom with the oxygen atom having an available bonding site for formation of an ether linkage.

The term “alkylene, as used herein, means an alkyl radical having two available bonding sites (i.e., -alkyl-), wherein “alkyl” is defined as above.

The term “alkenyl” is intended to include hydrocarbon chains of either a straight or branched configuration comprising one or more unsaturated carbon-carbon bonds which may occur in any stable point along the chain, such as ethenyl and propenyl. Alkenyl groups typically will have 2 to about 12 carbon atoms, more typically 2 to about 8 carbon atoms.

The term “aryl” is intended to include groups that, in accordance with the theory of Hückel, have a cyclic, delocalized (4n+2) pi-electron system. Examples of aryl groups include, but are not limited to, arenes and their substitution products, e.g. phenyl, naphthyl and toluyl, among numerous others.

The term “heteroaryl” is intended to include aromatic heterocyclic groups and includes the non-limiting examples furanyl, thiophene-yl, pyridyl, pyrimidyl, pyridazyl, oxazolyl, isooxazolyl, thiazolyl, thiadiazol and isothiazolyl, among others.

Unless otherwise indicated the term “heterocydoalkyl” as used herein includes a cyclic hydrocarbon in which one or more of the ring carbon atoms has been replaced with a nitrogen, oxygen or sulfur atom or any combination thereof and includes the non-limiting examples tetrahydrofuran, dioxane, morpholine, piperidine and pyrazine among others.

Unless otherwise indicated, the term “one or more substituents”, as used herein, refers to from one to the maximum number of substituents possible based on the number of available bonding sites.

The compounds of formula I may have chiral centers and therefore may occur in different enantiomeric configurations. The invention includes all enantiomers, diastereomers, and other stereoisomers of such compounds of formula I, as well as racemic and other mixtures thereof.

The present invention also relates to the pharmaceutically acceptable acid addition salts of the compounds of formula I. Examples of pharmaceutically acceptable acid addition salts of the compounds of formula I are the salts of hydrochloric acid, p-toluenesulfonic acid, fumaric acid, citric acid, succinic acid, salicylic acid, oxalic acid, hydrobromic acid, phosphoric acid, methanesulfonic acid, tartaric acid, malate, di-p-toluoyl tartaric acid, and mandelic acid.

The present invention also, relates to all radiolabeled forms of the compounds of the formula I. Preferred radiolabeled compounds of formula I are those wherein the radiolabels are selected from as ³H, ¹¹C, ¹⁴C, ¹⁸F, ¹²³I and ¹²⁵I. Such radiolabeled compounds are useful as research and diagnostic tools in metabolism pharmacokinetics studies and in binding assays in both animals and man.

The present invention also relates to a pharmaceutical composition for treating a disorder or condition in a mammal, including a human, selected from depression, anxiety, depression with concomitant anxiety, post traumatic stress disorder, panic phobias, obsessive compulsive disorder (OCD), borderline personality disorder, sleep disorder, psychosis, seizures, dyskinesis, symptoms of Huntington's or Parkinson's diseases, spasticity, suppression of seizures resulting from epilepsy, cerebral ischemia, anorexia, faintness attacks, hypokinesia, cranial traumas, chemical dependencies, premature ejaculation, premenstrual syndrome (PMS) associated mood and appetite disorder, inflammatory bowel disease, modification of feeding behavior, blocking carbohydrate cravings, late luteal phase dysphoric disorder, tobacco withdrawal-associated symptoms, panic disorder, bipolar disorder, sleep disorders, jet lag, cognitive dysfunction, hypertension, bulimia, anorexia, obesity, cardiac arrhythmias, chemical dependencies and addictions (e.g., dependencies on, or addictions to nicotine (and/or tobacco products), alcohol, benzodiazepines, barbiturates, opioids or cocaine), headache, stroke, traumatic brain injury (TBI), psychosis, Huntington's Chorea, tardive dyskinesia, hyperkinesia, dyslexia, schizophrenia, multi-infarct dementia, epilepsy, senile dementia of the Alzheimer's type (AD), Parkinson's disease (PD), attention deficit hyperactivity disorder (ADHD) and Tourette's Syndrome, comprising an amount of a compound of the formula I, or a pharmaceutically acceptable salt thereof, and a pharmaceutically acceptable carrier.

The present invention also relates to a method of treating a disorder or condition in a mammal, including a human, selected from depression, anxiety, depression with concomitant anxiety, post traumatic stress disorder, panic phobias, obsessive compulsive disorder (OCD), borderline personality disorder, sleep disorder, psychosis, seizures, dyskinesis, symptoms of Huntington's or Parkinson's diseases, spasticity, suppression of seizures resulting from epilepsy, cerebral ischemia, anorexia, faintness attacks, hypokinesia, cranial traumas, chemical dependencies, premature ejaculation, premenstrual syndrome (PMS) associated mood and appetite disorder, inflammatory bowel disease, modification of feeding behavior, blocking carbohydrate cravings, late luteal phase dysphoric disorder, tobacco withdrawal-associated symptoms, panic disorder, bipolar disorder, sleep disorders, jet lag, cognitive dysfunction, hypertension, bulimia, anorexia, obesity, cardiac arrhythmias, chemical dependencies and addictions (e.g., dependencies on, or addictions to nicotine (and/or tobacco products), alcohol, benzodiazepines, barbiturates, opioids or cocaine), headache, stroke, traumatic brain injury (TBI), psychosis, Huntington's Chorea, tardive dyskinesia, hyperkinesia, dyslexia, schizophrenia, multi-infarct dementia, epilepsy, senile dementia of the Alzheimer's type (AD), Parkinson's disease (PD), attention deficit hyperactivity disorder (ADHD) and Tourette's Syndrome, comprising administering to a mammal in need of such treatment an amount of a compound of the formula I, or a pharmaceutically acceptable salt thereof, that is effective in treating such disorder or condition.

### DETAILED DESCRIPTION OF THE INVENTION

Except where otherwise stated R¹, R², R³, R⁴, R⁵, R⁶, R⁷, R⁸, k, G¹, G², G³, G⁴, X, A, D, W, Y, Z, a, n, m and p in the reaction schemes and discussion that follow are defined as above. Each of the following reaction schemes apply to X defined as either C or O, even if only one definition of X is given in the scheme itself. Unless otherwise stated reaction conditions include an inert atmosphere commonly used in the art such as nitrogen or argon.

Scheme 1 refers to methods for the preparation of compounds of formula I wherein n is 1, A is G², m is 1, p is 1 and wherein the group D, W, Y and Z are independently C or N; and wherein R² is as defined above.

In step 1 of Scheme 1 the enamine-benzamide of formula V is prepared by treating the cyclic ketone of formula VI with an excess of the piperazine of formula VII in the presence of an amine, ketone condensation catalyst, preferably titanium tetrachloride, at about −50° C. to about −78° C., preferably about −78° C., in an ethereal solvent, preferably tetrahydrofuran (THF), stirring about 12 hours to about 16 hours at about 20° C. to about 25° C. The solution is then treated with ammonium hydroxide and a solvent such as ethyl acetate to form a precipitate which is then treated with a base such as sodium hydroxide and a solvent such as methylene chloride.

In step 2 of Scheme 1 a compound of the formula IV is prepared by treating the enamine-benzamide of formula V with a reducing agent, preferably sodium cyanoborohydride, in an ethereal solvent such as THF, at about 0° C., in the presence of an acid such as HCl.

In step 3 of Scheme 1 the primary amine of formula III is prepared by hydrolyzing the amide of formula IV by treating with a strong acid such as HCl at about 85° C. to about 95° C., preferably at about 90° C. for about 10 hours to about 14 hours, preferably about 12 hours.

In step 4 of Scheme 1 the compound of formula I, wherein n is 1 and A is G², is prepared by treating a mixture of the primary amine of formula III and a carboxylic acid of the formula II with a coupling agent such as 1,3 dicyclohexylcarbodiimide (DCC), or O-benzotriazole-N,N,N′,N′-tetramethyl-uronium-hexafluoro-phosphate (HBTU), preferably HBTU, in an anhydrous amide solvent such as dimethylformamide (DMF), dimethylacetamide (DMAc) or N-methylpyrrolidone (NMP), preferably DMF, followed by the addition of a tertiary amine, preferably triethylamine at about 50° C. to about 70° C., preferably at about 60° C. for about 15 hours to about 25 hours, preferably about 20 hours.

As will be evident to those skilled in the art compound I, of Scheme I, may be further treated to alter the nature of the functional group R2. For example, when R2 is a carboxylate ester, treatment with an alkyl magnesium halide will convert R2 to a dialkylcarbinol substituent.

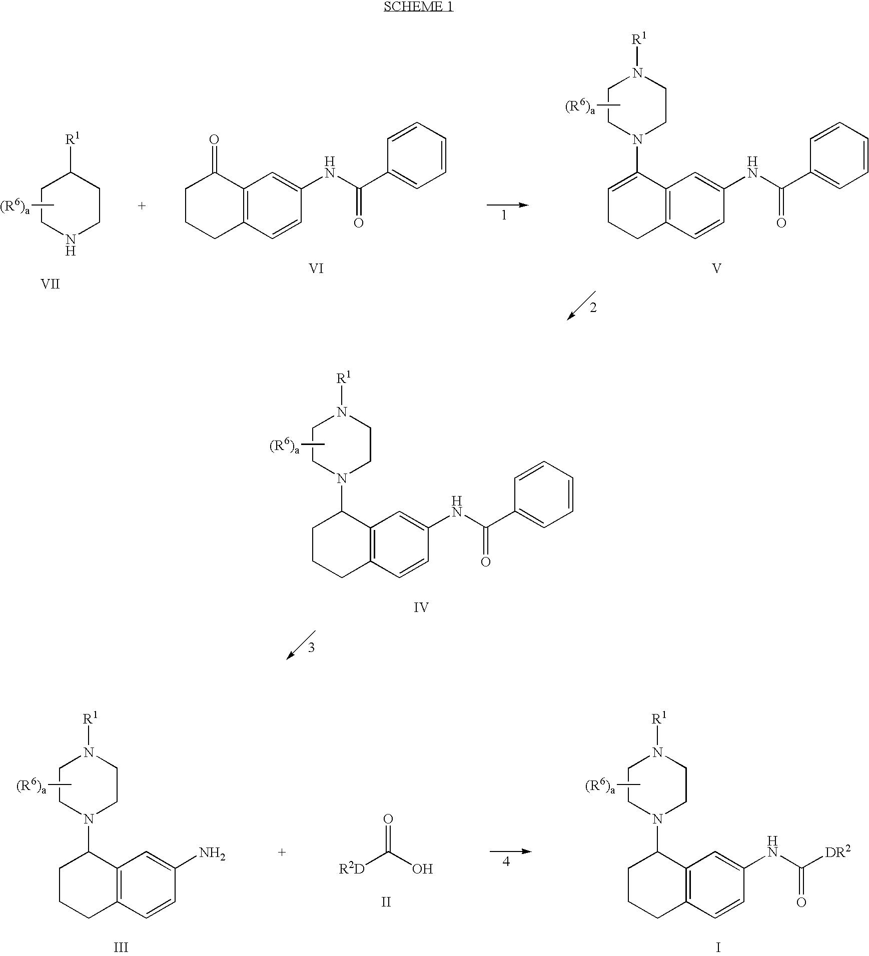

Scheme 2 refers to methods for the preparation of compounds of formula I wherein X is CH₂ or O, n is zero, m is 1 and p is 1, wherein the group D, W, Y and Z are independently C or N; and wherein R² is as defined above.

In step 1 of Scheme 2 the compound of formula XIII is prepared by treating the compound of the formula XIV with an excess of an ether bond cleaving agent such as aluminum trichloride in an aromatic solvent such as toluene at about 110° C. for about 30 minutes to about 75 minutes, preferably about 45 minutes and then cooled to about 0° C. and quenched with water.

In step 2 of Scheme 2 the compound of formula XII is prepared by treating the compound of formula XIII with a reducing agent, preferably sodium borohydride in an alcoholic solvent, preferably methanol, at about 0° C. for about 1.5 hours to about 2.5 hours, preferably about 2 hours.

In step 3 of Scheme 2 the compound of formula XI, wherein the alicyclic hydroxyl of the compound of formula XII is replaced with a leaving group, preferably a halogen, is prepared by treating the compound of formula XII with a halogenating agent, preferably thionyl chloride in a solvent, preferably an aromatic solvent such as toluene at about 50° C. to about 60° C., preferably about 55° C., for about 1 hour to about 2 hours, preferably about 1 hour, and then cooling and quenching with water.

In step 4 of Scheme 2 the compound of formula X is prepared by treating the compound of formula XI with the piperazine of formula VII in the presence of a base such as potassium carbonate and a catalyst such as sodium iodide in a solvent such as acetonitile, at about 80° C. to about 90° C., preferably about 85° C., for about 2 hours to about 3 hours, preferably about 2 hours.

In step 5 of Scheme 2 the compound of formula IX is prepared by treating the compound of formula X with a strong base such as sodium hexamethyldisilazane (NaHMDS) in an ethereal solvent, preferably THF at about −78° C. for about 20 minutes to about 40 minutes, preferably about 30 minutes, and then adding a triflating agent such as N-phenyl trifluoromethanesulfonimide (PhNTf₂) while continuing to maintain a temperature of about −780° C. for an additional period of about 10 minutes to about 30 minutes, preferably about 20 minutes and then warming to about room temperature.

In step 6 of Scheme 2 the compound of formula I, wherein n is zero and the group A is absent, is prepared by standard Suzuki coupling conditions such as treating the compound of formula IX with the boronic acid of formula VIII, in a mixture of an alcohol, preferably ethanol, water and an such as dimethoxyethane (DME) in the presence of cesium carbonate and Pd(Ph₃P)₄ at about 80° C. to about 100° C., preferably about 90° C., for about 14 hours to about 24 hours, preferably about 19 hours.

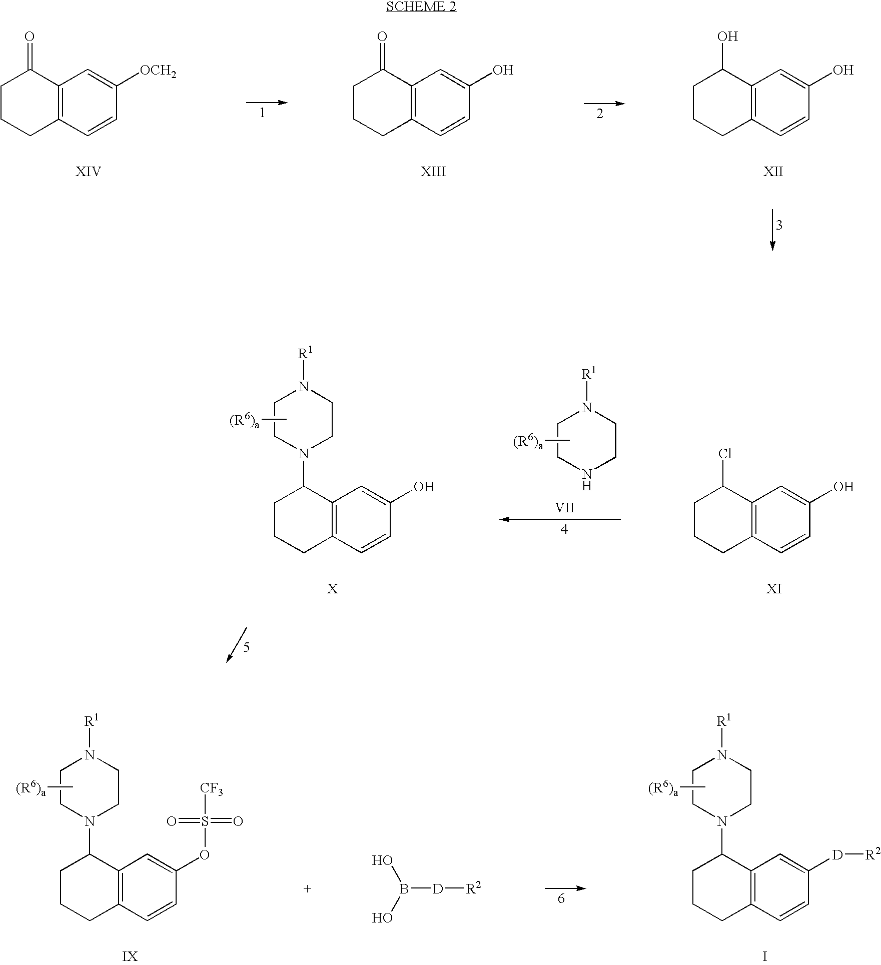

Scheme 3 refers to methods for the preparation of compounds of formula I wherein n is 1, A is G¹, m is zero and p is one.

In step 1 of Scheme 3 the compound of formula XV is prepared by treating a mixture of the compound of formula IX, prepared as described in Scheme 2, Pd(OAc)₂, 1,3-bis(diphenylphosphino)propene, and a tertiary amine, preferably triethylamine, and dimethylsulfoxide (DMSO) in an alcoholic solvent, preferably methanol with CO at about 45 psi to about 55 psi, preferably 50 psi, at about 65° C. to about 75° C., preferably about 70° C. for about 12 hours to about 20 hours, preferably about 16 hours.

In step 2 of Scheme 3 the compound of formula I wherein n is 1 and the group A is G¹ is prepared by treating an amine of the formula XX with an alkyl aluminum such as trimethylaluminum in a reaction inert solvent, preferably dichloromethane and toluene, for about 30 minutes at about 20° C. to about 25° C. and then adding a solution of the compound of formula XV in a reaction inert solvent, preferably dichloromethane, and heating at about 45° C. to about 55° C., preferably about 50° C. for about 14 hours to about 24 hours, preferably about 19 hours.

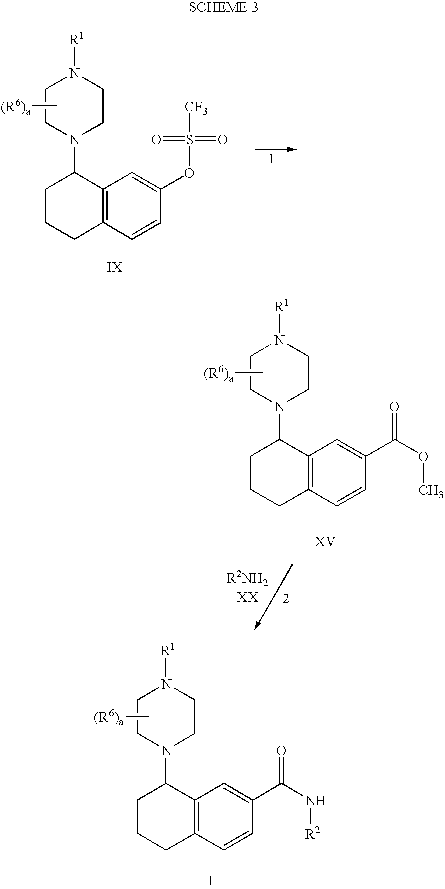

Scheme 4 refers to methods for the preparation of compounds of formula I wherein n is zero, m is one, p is one and wherein the group D, W, Y and Z are independently C or N; and wherein R² is as defined above, wherein a tetrahydronaphthyl boronic ester is coupled to an aryl or heteroaryl halide of the formula XXI.

In step 1 of Scheme 4 the compound of formula XVI is prepared by treating the compound of formula IX, prepared as described in Scheme 2, with bis(pinocolato)diboron, Pd(dppf)Cl₂CH₂.Cl₂ and potassium acetate in an anhydrous amide solvent, preferably DMF, at about 750° C. to about 85° C., preferably about 80° C., for about 3 hours to about 5 hours, preferably about 4 hours.

In step 2 of Scheme 4 the compound of formula I is prepared by treating the compound of formula XVI, with an aryl or heteroaryl halide, preferably an iodide or bromide of formula XXI, with Pd(dppf)Cl₂.CH₂Cl₂ catalyst and sodium carbonate in water containing an amide solvent, preferably DMF at about 75° C. to about 85° C., preferably about 80° C., for about 12 hours to about 20 hours, preferably about 16 hours.

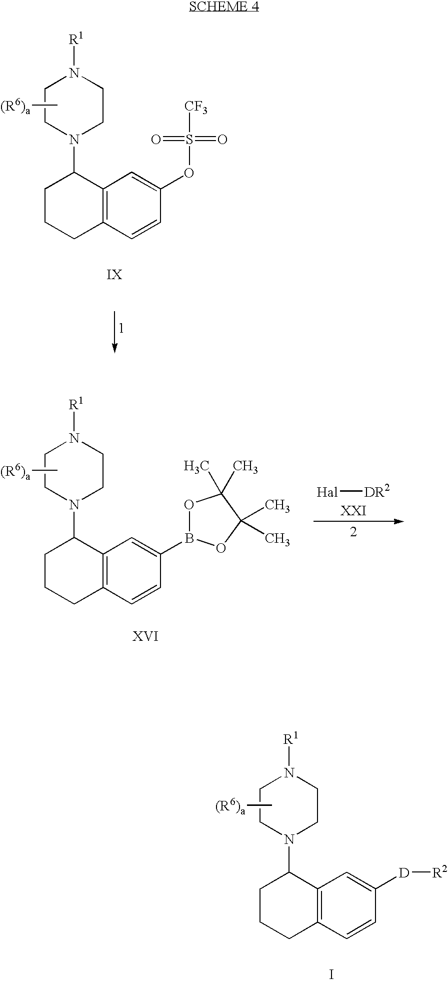

Scheme 5 refers to methods for the preparation of compounds of formula I wherein X is O or C, n is zero, m is one, p is one and wherein the group D, W, Y and Z are independently C or N; and wherein R² is as defined above, wherein a boronic ester is coupled to an aryl or heteroaryl halide of the formula XXI.

In step 1 of Scheme 5 the compound of formula XXIII is prepared by treating the compound of the formula XXII with thionyl chloride in a reaction inert solvent such as an aromatic solvent, preferably toluene at about 35° C. to about 45° C., preferably about 40° C. for about 30 minutes to about 90 minutes, preferably about 60 minutes.

In step 2 of Scheme 5 the compound of formula XXIV is prepared by treating the compound of the formula XXIII with the piperazine of formula VII in an anhydrous polar solvent such as acetonitrile, in the presence of a base such as potassium carbonate and sodium iodide at about 65° C. to about 75° C., preferably about 70° C. for about 12 hours to about 20 hours, preferably about 16 hours.

In step 3 of Scheme 5 the compound of formula XXV is prepared by treating the compound of formula XXIV with bis(pinocolato)diboron, potassium carbonate and Pd(dppf)Cl₂.CH₂Cl₂ in an anhydrous amide solvent, preferably DMF, at about 75° C. to about 85° C., preferably about 80° C., for about 3 hours to about 5 hours, preferably about 4 hours.

In step 4 of Scheme 5 the compound of formula I, wherein X is 0, is prepared by treating the compound of formula XXV, with an aryl or heteroaryl halide, preferably a bromide of formula XXI, with Pd(dppf)Cl₂.CH₂Cl₂ catalyst and sodium carbonate in water containing an amide solvent, preferably DMF at about 75° C. to about 85° C., preferably about 80° C., for about 12 hours to about 20 hours, preferably about 16 hours. In step 5 of Scheme 5 the compound of formula I is prepared by treating the compound of formula XXIV, is prepared by treating the compound of formula XXIV with the boronic acid of formula VII, in a mixture of an alcohol, preferably ethanol, water and an such as dimethoxyethane (DME) in the presence of cesium carbonate and Pd(Ph₃P)₄ at about 80° C. to about 100° C., preferably about 90° C., for about 14 hours to about 24 hours, preferably about 19 hours.

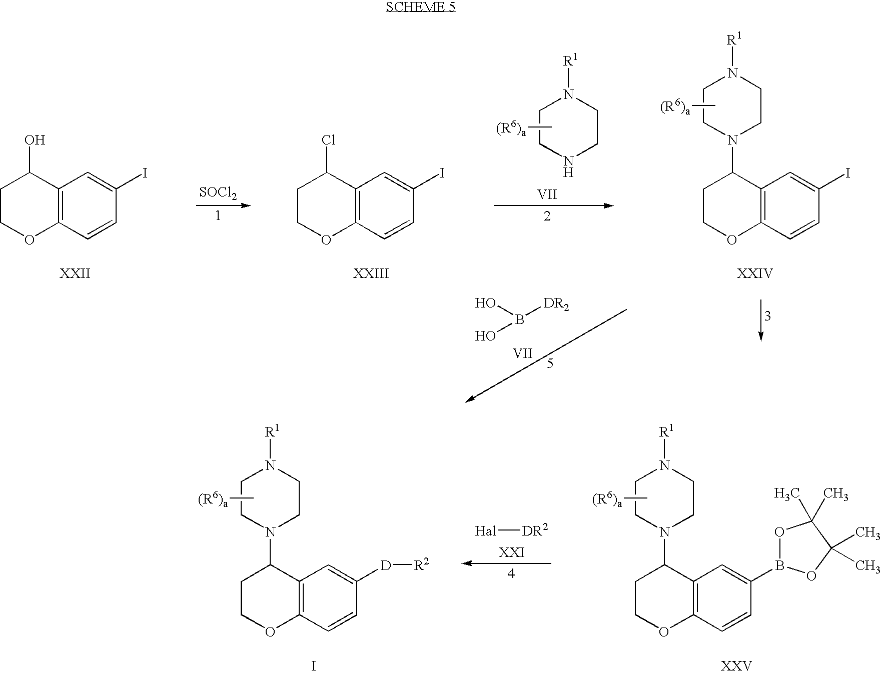

Scheme 6 refers to methods for the preparation of compounds of formula I wherein n is 1, A is G⁴, m is 1, p is 1 and wherein the group D, W, Y and Z are independently C or N; and wherein R² is as defined above.

In step 1 of Scheme 6 the compound of formula XXVII is prepared by treating the compound of the formula XI, prepared as described in Scheme 2, with an N-protected piperazine compound of formula XXVI, such as 1-t-butylpiperzine carboxylate in the presence of a base such as potassium carbonate, and sodium iodide in an anhydrous polar solvent such as acetonitrile at about 75° C. to about 85° C., preferably about 80° C. for about 12 hours to about 20 hours, preferably about 12 hours.

In step 2 of Scheme 6 the compound of formula XXIX is prepared by treating the compound of the formula XXVII with a compound of the formula XXVIII, wherein L is a group readily displaced by a nucleophile, preferably bromo, in a solvent such as acetone, in the presence of an alkali metal base such as potassium carbonate, at about 45° C. to about 55° C., preferably about 50° C. for about 20 hours to about 24 hours, preferably about 22 hours and then stirring at about 20° C. to about 25° C. for about 14 hours to about 18 hours.

In step 3 of Scheme 6, which is the reduction of the nitrile group to a primary amine, the compound of formula XXX is prepared by treating the compound of the formula XXIX with a reducing agent, preferably lithium aluminum hydride (LAH), in an ethereal solvent, preferably ether at about −5° C. to about 5° C., preferably about 0° C., warming to about 20° C. to about 25° C. for about 30 minutes, and then cooling to about 0° C. and quenching with an alkali metal base such as NaOH.

In step 4 of Scheme 6, the compound of the formula XXXII is prepared by treating the compound of the formula XXX with the compound of the formula XXXI wherein L is a group readily displaced by a nucleophile, preferably halo, and D and R² are as defined above, in an anhydrous polar solvent, preferably DMF, in the presence of an alkali metal base such as sodium bicarbonate at about 90° C. to about 100° C., preferably about 95° C. for about 12 hours to about 20 hours, preferably about 16 hours.

Step 5 of Scheme 6 is a deprotection step wherein the compound of formula Ia wherein R¹ is H is prepared by removal of protective group P from the compound of formula XXXII. When protective group P is t-Boc compound XXXII is typically treated with a strong acid such as HCl in an anhydrous reaction inert solvent such as a mixture of a chlorinated hydrocarbon and an ether, preferably methylene chloride and ethyl ether at about 20° C. to about 25° C. for about 10 hours to about 14 hours, preferably about 12 hours.

In step 5a of Scheme 6, the compound of formula I, wherein R¹ is methyl is prepared directly from the compound of formula XXXII, wherein P is the protective group t-Boc, by treating with lithium aluminum hydride in an ethereal solvent, preferably THF at about 45° C. to about 65° C., preferably about 55° C. for about 5 hours to about 58 hours, preferably about 48 hours.

In step 6 of Scheme 6 compound I wherein R¹ is alkyl is prepared from the compound of formula Ia by alkylation methods known in the art. Compound I, wherein R¹ is methyl is prepared by treating compound Ia in an ethereal solvent, preferably THF with a formic acid, formalin mixture at about 75° C. to about 85° C., preferably about 80° C., for about 2 hours to about 4 hours, preferably about 3 hours and then slowly cooling to about 20° C. to about 25° C.

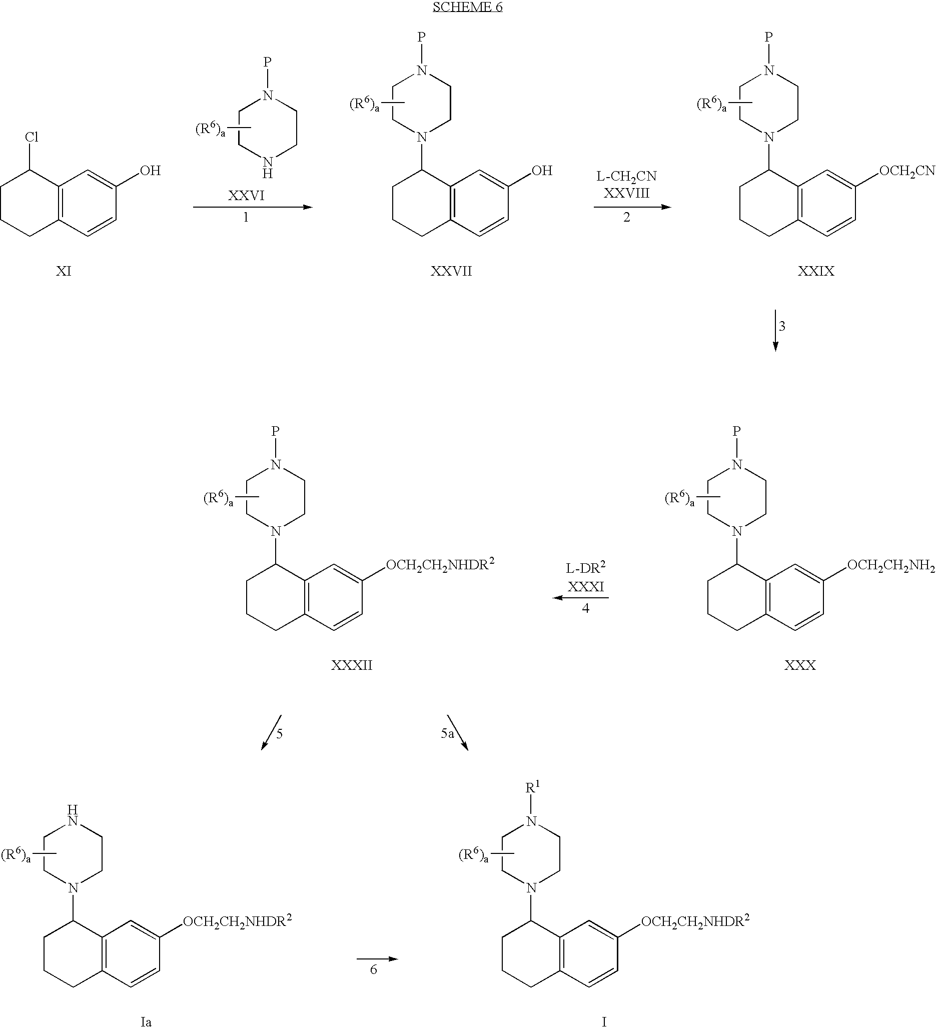

Scheme 7 refers to methods for the preparation of compounds of formula I wherein n is one, A is G³, m is one and p is zero, and wherein group D, W, Y and Z are independently C or N.

In step 1 of Scheme 7 the compound of formula XXXIV is prepared by treating the compound of formula XXVII, prepared as described in Scheme 6, with a benzylic halide, preferably a bromide, of the formula XXXIII, in the presence of an alkali metal base, preferably a carbonate, most preferably cesium carbonate, in an anhydrous polar solvent such as acetonitrile at about 50° C. to about 70° C., preferably about 60° C. for about 8 hours to about 16 hours, preferably about 12 hours.

Step 2 of Scheme 7 is a deprotection step wherein the compound of formula Ib wherein R¹ is H is prepared by removal of protective group P from the compound of formula XXXII. When protective group P is t-Boc compound XXXII is typically treated with a strong acid such as HCl in an anhydrous reaction inert solvent such as a mixture of a chlorinated hydrocarbon and an ether, preferably methylene chloride and ethyl ether at about 20° C. to about 25° C. for about 10 hours to about 14 hours, preferably about 12 hours.

In step 2a of Scheme 7, the compound of formula I, wherein R¹ is methyl is prepared directly from the compound of formula XXXIV, wherein P is the protective group t-Boc, by treating with lithium aluminum hydride in an ethereal solvent, preferably THF at about 45° C. to about 65° C., preferably about 55° C. for about 38 hours to about 58 hours, preferably about 48 hours.

In step 3 of Scheme 7 compound I wherein R¹ is alkyl is prepared from the compound of formula Ia by alkylation methods known in the art such as treatment with an aldehyde and reduction. Compound I, wherein R¹ is methyl is also prepared by treating compound Ib in an ethereal solvent, preferably THF with a formic acid, formalin mixture at about 75° C. to about 85° C., preferably about 80° C., for about 2 hours to about 4 hours, preferably about 3 hours and then slowly cooling to about 20° C. to about 25° C.

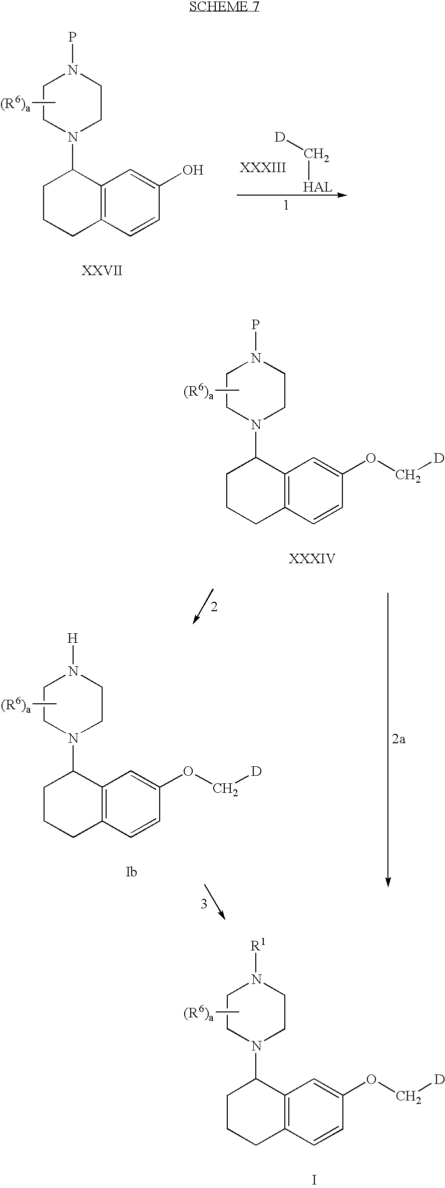

Scheme 8 refers to methods for the preparation of compounds of formula I wherein n is one, A is G⁶, wherein R⁷ is defined as above, m is one or zero, p is one and wherein group D, the ring members represented by W, Y and Z are independently C or N; and wherein R² is as defined above.

In step 1 of Scheme 8 the compound of formula XXXV is prepared by treating the compound of formula XVI with a pyridyl halide XXIa in the manner described in step 2 of Scheme 4.

In step 2 of Scheme 8 the compound of formula XXXVI is prepared by catalytic reduction of the compound of formula XXXV, preferably with PtO₂, in a solvent such as acetic acid, under a hydrogen pressure of about 30-80 psi for about 1 to 24 hours.

In step 3 of Scheme 8 a compound of the formula I-G⁶, wherein n is 1, m is 0 and p is one is prepared by treating the compound of the formula XXXVI with a compound of the formula XXXVII, typically an aryl carboxylic acid, and a coupling agent such as 1,3 dicyclohexylcarbodiimide (DCC), or O-benzotriazole-N,N,N′,N′-tetramethyl-uronium-hexafluoro-phosphate (HBTU), preferably HBTU, in methylene chloride containing an anhydrous amide solvent such as dimethylformamide (DMF), dimethylacetamide (DMAc) or N-methylpyrrolidone (NMP), preferably DMF, in the presence of a tertiary amine, preferably triethylamine at about 50° C. to about 70° C., preferably at about 60° C. for about 3 hours to about 5 hours, preferably about 4 hours.

In step 3a of Scheme 8 a compound of the formula I-G^{6A}, wherein n is 1, m is 1 and p is 1 is prepared by treating the compound of the formula XXXVI with an aryl or heteroaryl halide, preferably a bromide, of the formula XXI, in an anhydrous aromatic solvent, preferably anhydrous toluene with Pd(OAc)₂ and racemic BINAP at about 95° C. to about 105° C., preferably at about 100° C. for about 14 hours to about 18 hours, preferably about 16 hours.

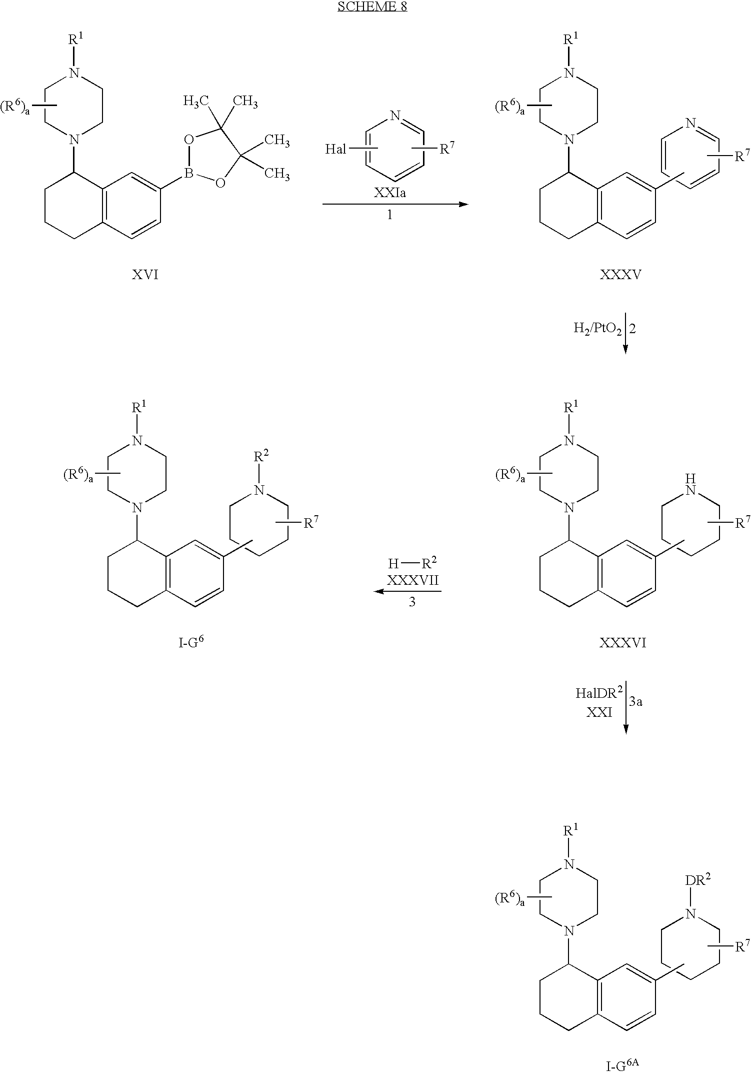

Scheme 9 refers to methods for the preparation of compounds of formula I wherein, n is one, A is G⁵, m is one, p is one and wherein group D, the ring members represented by W, Y and Z are independently C or N; and wherein R² is as defined above.

In step 1 of Scheme 9 a compound of the formula IG⁵ is prepared by treating a compound of formula X, prepared as described in Scheme 2, with the carboxylic acid of formula II, in the presence of a coupling agent such as DCC, preferably in the presence of an acylation catalyst such as DMAP, in an anhydrous reaction inert solvent such as methylene chloride or ethyl ether, preferably methylene chloride, at about 20° C. to about 25° C. for about 12 to about 20 hours, preferably about 16 hours.

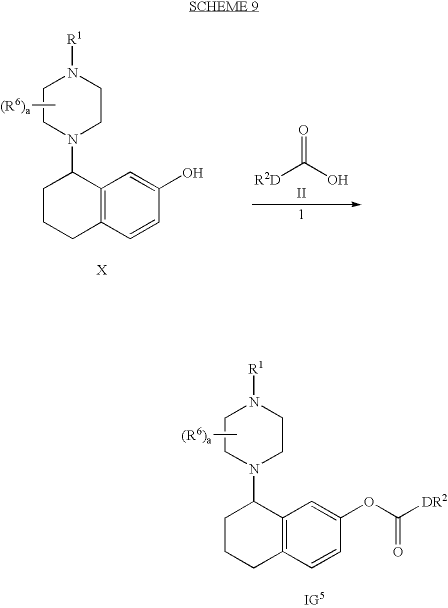

The compounds of the formula I and their pharmaceutically acceptable salts (hereafter “the active compounds”) can be administered via either the oral, transdermal (e.g., through the use of a patch), intranasal, sublingual, rectal, parenteral or topical routes. Transdermal and oral administration are preferred. These compounds are, most desirably, administered in dosages ranging from about 0.25 mg up to about 1500 mg per day, preferably from about 0.25 to about 300 mg per day in single or divided doses, although variations will necessarily occur depending upon the weight and condition of the subject being treated and the particular route of administration chosen. However, a dosage level that is in the range of about 0.01 mg to about 10 mg per kg of body weight per day is most desirably employed. Variations may nevertheless occur depending upon the weight and condition of the persons being treated and their individual responses to said medicament, as well as on the type of pharmaceutical formulation chosen and the time period and interval during which such administration is carried out. In some instances, dosage levels below the lower limit of the aforesaid range may be more than adequate, while in other cases still larger doses may be employed without causing any harmful side effects, provided that such larger doses are first divided into several small doses for administration throughout the day.

The active compounds can be administered alone or in combination with pharmaceutically acceptable carriers or diluents by any of the several routes previously indicated. More particularly, the active compounds can be administered in a wide variety of different dosage forms, e.g., they may be combined with various pharmaceutically acceptable inert carriers in the form of tablets, capsules, transdermal patches, lozenges, troches, hard candies, powders, sprays, creams, salves, suppositories, jellies, gels, pastes, lotions, ointments, aqueous suspensions, injectable solutions, elixirs, syrups, and the like. Such carriers include solid diluents or fillers, sterile aqueous media and various non-toxic organic solvents. In addition, oral pharmaceutical compositions can be suitably sweetened and/or flavored. In general, the active compounds are present in such dosage forms at concentration levels ranging from about 5.0% to about 70% by weight.

For oral administration, tablets containing various excipients such as microcrystalline cellulose, sodium citrate, calcium carbonate, dicalcium phosphate and glycine may be employed along with various disintegrants such as starch (preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc can be used for tabletting purposes. Solid compositions of a similar type may also be employed as fillers in gelatin capsules; preferred materials in this connection also include lactose or milk sugar] as well as high molecular weight polyethylene glycols. When aqueous suspensions and/or elixirs are desired for oral administration the active ingredient may be combined with various sweetening or flavoring agents, coloring matter and, if so desired, emulsifying and/or suspending agents, together with such diluents as water, ethanol, propylene glycol, glycerin and various combinations thereof.

For parenteral administration, a solution of an active compound in either sesame or peanut oil or in aqueous propylene glycol can be employed. The aqueous solutions should be suitably buffered (preferably pH greater than 8), if necessary, and the liquid diluent first rendered isotonic. These aqueous solutions are suitable for intravenous injection purposes. The oily solutions are suitable for intraarticular, intramuscular and subcutaneous injection purposes. The preparation of all these solutions under sterile conditions is readily accomplished by standard pharmaceutical techniques well known to those skilled in the art.

It is also possible to administer the active compounds topically and this can be done by way of creams, a patch, jellies, gels, pastes, ointments and the like, in accordance with standard pharmaceutical practice.

### Biological Assay

The activity of the compounds of the present invention with respect to 5HT_{1B} (formerly 5HT_{1D}) binding ability can be determined using standard radioligand binding assays as described in the literature. The 5-HT_{1A} affinity can be measured using the procedure of Hoyer et al. (Brain Res., 376, 85 (1986)). The 5-HT_{1D} affinity can be measured using the procedure of Heuring and Peroutka (J. Neurosci., 7, 894 (1987)).

The in vitro activity of the compounds of the present invention at the 5-HT_{1D} binding site may be determined according to the following procedure. Bovine caudate tissue is homogenized and suspended in 20 volumes of a buffer containing 50 mM TRIS hydrochloride (tris[hydroxymethyl]aminomethane hydrochloride) at a pH of 7.7. The homogenate is then centrifuged at 45,000 G for 10 minutes. The supernatant is then discarded and the resulting pellet resuspended in approximately 20 volumes of 50 mM TRIS hydrochloride buffer at pH 7.7. This suspension is then pre-incubated for 15 minutes at 37° C., after which the suspension is centrifuged again at 45,000 G for 10 minutes and the supernatant discarded. The resulting pellet (approximately 1 gram) is resuspended in 150 ml of a buffer of 15 mM TRIS hydrochloride containing 0.01 percent ascorbic acid with a final pH of 7.7 and also containing 10 μM pargyline and 4 mM calcium chloride (CaCl₂). The suspension is kept on ice at least 30 minutes prior to use.

The inhibitor, control or vehicle is then incubated according to the following procedure. To 50 μl of a 20 percent dimethylsulfoxide (DMSO)/80 percent distilled water solution is added 200 μl of tritiated 5-hydroxytryptamine (2 nM) in a buffer of 50 mM TRIS hydrochloride containing 0.01 percent ascorbic acid at pH 7.7 and also containing 10 μM pargyline and 4 μM calcium chloride, plus 100 nM of 8-hydroxy-DPAT (dipropylaminotetraline) and 100 nM of mesulergine. To this mixture is added 750 μl of bovine caudate tissue, and the resulting suspension is vortexed to ensure a homogenous suspension. The suspension is then incubated in a shaking water bath for 30 minutes at 25° C. After incubation is complete, the suspension is filtered using glass fiber filters (e.g., Whatman GF/B™ filters.). The pellet is then washed three times with 4 ml of a buffer of 50 mM TRIS hydrochloride at pH 7.7. The pellet is then placed in a scintillation vial with 5 ml of scintillation fluid (Aquasol 2™) and allowed to sit overnight. The percent inhibition can be calculated for each dose of the compound. An IC₅₀ value can then be calculated from the percent inhibition values.

The activity of the compounds of the present invention for 5-HT_{1A} binding ability can be determined according to the following procedure. Rat brain cortex tissue is homogenized and divided into samples of 1 gram lots and diluted with 10 volumes of 0.32 M sucrose solution. The suspension is then centrifuged at 900 G for 10 minutes and the supernate separated and recentrifuged at 70,000 G for 15 minutes. The supernate is discarded and the pellet re-suspended in 10 volumes of 15 mM TRIS hydrochloride at pH 7.5. The suspension is allowed to incubate for 15 minutes at 37° C. After pre-incubation is complete, the suspension is centrifuged at 70,000 G for 15 minutes and the supernate discarded. The resulting tissue pellet is resuspended in a buffer of 50 mM TRIS hydrochloride at pH 7.7 containing 4 mM of calcium chloride and 0.01 percent ascorbic acid. The tissue is stored at −70° C. until ready for an experiment. The tissue can be thawed immediately prior to use, diluted with 10 μm pargyline and kept on ice.

The tissue is then incubated according to the following procedure. Fifty microliters of control, inhibitor, or vehicle (1 percent DMSO final concentration) is prepared at various dosages. To this solution is added 200 μl of tritiated DPAT at a concentration of 1.5 nM in a buffer of 50 mM TRIS hydrochloride at pH 7.7 containing 4 mM calcium chloride, 0.01 percent ascorbic acid and pargyline. To this solution is then added 750 μl of tissue and the resulting suspension is vortexed to ensure homogeneity. The suspension is then incubated in a shaking water bath for 30 minutes at 37° C. The solution is then filtered, washed twice with 4 ml of 10 mM TRIS hydrochloride at pH 7.5 containing 154 mM of sodium chloride. The percent inhibition is calculated for each dose of the compound, control or vehicle. IC₅₀ values are calculated from the percent inhibition values.

The agonist and antagonist activities of the compounds of the invention at 5-HT_{1A} and 5-HT₁ receptors can be determined using a single saturating concentration according to the following procedure. Male Hartley guinea pigs are decapitated and 5-HT_{1A} receptors are dissected out of the hippocampus, while 5-HT_{1D} receptors are obtained by slicing at 350 mM on a Mcllwain tissue chopper and dissecting out the substantia nigra from the appropriate slices. The individual tissues are homogenized in 5 mM HEPES buffer containing 1 mM EGTA (pH 7.5) using a hand-held glass-Teflon® homogenizer and centrifuged at 35,000×g for 10 minutes at 4° C. The pellets are resuspended in 100 mM HEPES buffer containing 1 mM EGTA (pH 7.5) to a final protein concentration of 20 mg (hippocampus) or 5 mg (substantia nigra) of protein per tube. The following agents are added so that the reaction mix in each tube contained 2.0 mM MgCl₂, 0.5 mM ATP, 1.0 mM CAMP, 0.5 mM IBMX, 10 mM phosphocreatine, 0.31 mg/mL creatine phosphokinase, 100 μM GTP and 0.5-1 microcuries of [³²P]-ATP (30 Ci/mmol: NEG-003—New England Nuclear). Incubation is initiated by the addition of tissue to siliconized microfuge tubes (in triplicate) at 30° C. for 15 minutes. Each tube receives 20 μL tissue, 10 μL drug or buffer (at 10× final concentration), 10 μL 32 nM agonist or buffer (at 10× final concentration), 20 μL forskolin (3 μM final concentration) and 40 μL of the preceding reaction mix. Incubation is terminated by the addition of 100 μL 2% SDS, 1.3 mM CAMP, 45 mM ATP solution containing 40,000 dpm [³H]-CAMP (30 Ci/mmol: NET-275—New England Nuclear) to monitor the recovery of CAMP from the columns. The separation of [³²P]-ATP and [³²P]-CAMP is accomplished using the method of Salomon et al., Analytical Biochemistry, 1974, 58, 541-548. Radioactivity is quantified by liquid scintillation counting. Maximal inhibition is defined by 10 μM (R)-8-OH-DPAT for 5-HT_{1A} receptors, and 320 nM 5-HT for 5-HT_{1D} receptors. Percent inhibitions by the test compounds are then calculated in relation to the inhibitory effect of (R)-8-OH-DPAT for 5-HT_{1A} receptors or 5-HT for 5-HT_{1D} receptors. The reversal of agonist induced inhibition of forskolin-stimulated adenylate cyclase activity is calculated in relation to the 32 nM agonist effect.

The compounds of the invention can be tested for in vivo activity for antagonism of 5-HT_{1D} agonist-induced hypothermia in guinea pigs according to the following procedure.

Male Hartley guinea pigs from Charles River, weighing 250-275 grams on arrival and 300-600 grams at testing, serve as subjects in the experiment. The guinea pigs are housed under standard laboratory conditions on a 7 a.m. to 7 p.m. lighting schedule for at least seven days prior to experimentation. Food and water are available ad libitum until the time of testing.

The compounds of the invention can be administered as solutions in a volume of 1 ml/kg. The vehicle used is varied depending on compound solubility. Test compounds are typically administered either sixty minutes orally (p.o.) or 0 minutes subcutaneously (s.c.) prior to a 5-HT_{1D} agonist, such as [3-(1-methylpyrrolidin-2-ylmethyl)-1H-indol-5-yl]-(3-nitropyridin-3-yl)-amine, which can be prepared as described in PCT publication WO93/11106, published Jun. 10, 1993 which is administered at a dose of 5.6 mg/kg, s.c. Before a first temperature reading is taken, each guinea pig is placed in a clear plastic shoe box containing wood chips and a metal grid floor and allowed to acclimate to the surroundings for 30 minutes. Animals are then returned to the same shoe box after each temperature reading. Prior to each temperature measurement each animal is firmly held with one hand for a 30-second period. A digital thermometer with a small animal probe is used for temperature measurements. The probe is made of semi-flexible nylon with an epoxy tip. The temperature probe is inserted 6 cm. into the rectum and held there for 30 seconds or until a stable recording is obtained. Temperatures are then recorded.

In p.o. screening experiments, a “pre-drug” baseline temperature reading is made at −90 minutes, the test compound is given at −60 minutes and an additional −30 minute reading is taken. The 5-HT_{1D} agonist is then administered at 0 minutes and temperatures are taken 30, 60, 120 and 240 minutes later. In subcutaneous screening experiments, a pre-drug baseline temperature reading is made at −30 minutes. The test compound and 5-HT_{1D} agonists are given concurrently and temperatures are taken at 30, 60, 120 and 240 minutes later.

Data are analyzed with two-way analysis of variants with repeated measures in Newman-Keuls post hoc analysis.

The active compounds of the invention can be evaluated as anti-migraine agents by testing the extent to which they mimic sumatriptan in contracting the dog isolated saphenous vein strip (P. P. A. Humphrey et al., Br. J. Pharmacol., 94, 1128 (1988)). This effect can be blocked by methiothepin, a known serotonin antagonist. Sumatriptan is known to be useful in the treatment of migraine and produces a selective increase in carotid vascular resistance in the anesthetized dog. The pharmacological basis of sumatriptan efficacy has been discussed in W. Fenwick et al., Br. J. Pharmacol., 96, 83 (1989).

The serotonin 5-HT₁ agonist activity can be determined by the in vitro receptor binding assays, as described for the 5-HT_{1A} receptor using rat cortex as the receptor source and [³H]-8-OH-DPAT as the radioligand (D. Hoyer et al. Eur. J. Pharm., 118, 13 (1985)) and as described for the 5-HT_{1D} receptor using bovine caudate as the receptor source and [³H]serotonin as the radioligand (R. E. Heuring and S. J. Peroutka, J. Neuroscience, 7, 894 (1987)). All compounds had IC₅₀ values of equal to or less than 500 nM.

The following experimental preparations and examples illustrate, but do not limit the scope of, this invention.

### Preparation of Intermediates

### Intermediate 1

### N-[8-(4-Methyl-piperazin-1-yl)-5,6-dihydro-naphthalen-2-yl]-benzamide

To a solution of N-methylpiperazine (250 mmol) and N-(8-Oxo-5,6,7,8-tetrahydro-naphthalen-2-yl)-benzamide (83 mmol) in 250 mL of tetrahydrofuran at −78° C. is added titanium tetrachloride (100 mL of a 1.0M solution in methylene chloride). The thick solid is stirred overnight with a mechanical stirrer at room temperature. To the solution is added 220 mL of ammonium hydroxide and 250 mL of ethyl acetate and stirred for 2 hours. The solid is stirred with 200 mL of 1N sodium hydroxide and 200 mL of methylene chloride for 7 hours, filtered, and the resultant solid dried in vacuo. The solid is then suspended in DMSO, washed with DMSO and filtered. The DMSO layers are combined and concentrated and then 250 mL of methylene chloride and 50 mL of brine is added. The resultant crystals are filtered and dried to provide 21.2 g of the title compound.

### Intermediate 2

### N-[8-(4-Methyl-piperazin-1-yl)-5,6,7,8-tetrahydro-naphthalen-2-yl]-benzamide

To a solution of N-[8-(4-Methyl-piperazin-1-yl)-5,6-dihydro-naphthalen-2-yl]-benzamide (5.3 mmol) in 10 mL of methanol at 0° C. is added sodiumcyanoborohydride (1.0 M solution in THF, 11 mL) and 10 mL of 1N HCl. The solvent is removed in vacuo, extracted with methylene chloride and brine, dried, and concentrated in vacuo. The crude material is used in the following step without further purification.

### Intermediate 3

### 8-(4-Methyl-piperazin-1-yl)-5,6,7,8-tetrahydro-naphthalen-2-ylamine

Intermediate 2 (22.92 mmol) was dissolved in 50.0 mL of EtOH and 3.0 mL of concentrated HCl was added. The resultant solution was heated at 90° C. for 12 hours. Upon cooling, the reaction was concentrated under reduced pressure and the residual oil was partitioned between 20 mL of 1N NaOH and 50.0 mL of dichloromethane. The organics were extracted and dried over MgSO₄, filtered and concentrated under reduced pressure. Purification was accomplished through flash chromatography on silica gel eluting with a gradient system of 100% CH₂Cl₂ to 15% CH₃OH/CH₂Cl₂ to 25% CH₃OH/CH₂Cl₂. Product containing fractions were combined and concentrated under reduced pressure to give the desired material as a colorless solid (5.62 g, 22.90 mmol, 100% yield. MS 246.2.

### Intermediate 4

### 7-Hydroxy-3,4-dihydro-2H-naphthalen-1-one

Aluminum trichloride (567.0 mmol) was dissolved in 500 mL of anhydrous toluene and 7-Methoxy-3,4-dihydro-2H-naphthalen-1-one (284.0 mmol) was added. The reaction was heated to 110° C. for 0.75 hour and then cooled. The reaction was further cooled to 0° C. in an ice bath and quenched with H₂O. The resultant solution was partitioned between H₂O and EtOAc. The organics were extracted and dried over MgSO₄, filtered and concentrated under reduced pressure. Purification was accomplished through trituration with CH₃OH to give the desired product (37.5 g, 231.0 mmol, 41% yield); MS 163.2 [M+H].

### Intermediate 5

### 1,2,3,4-Tetrahydro-naphthalene-1,7-diol

Intermediate 4 (21.58 mmol) was dissolved in 100 mL of methanol and cooled to 0° C. in an ice water bath. Sodium borohydride (43.16 mmol) was added and the reaction was stirred for 2 hours. The solvent was removed under reduced pressure and the residual oil was partitioned between 200 mL Et₂O and 35 mL H₂O. The organics were extracted, dried over MgSO₄, filtered and concentrated under reduced pressure to give the desired product (3.23 g, 19.6 mmol, 91% yield).

### Intermediate 6

### 8-Chloro-5,6,7,8-tetrahydro-naphthalen-2-ol

Intermediate 5 (6.09 mmol) was dissolved in 15 mL of anhydrous toluene and thionyl chloride (12.78 mmol) was added. The resultant solution was heated to 55° C. for 1.5 hours. Upon cooling, the reaction is slowly quenched by the drop wise addition of H₂O. The organics were extracted and dried over MgSO₄, filtered and concentrated under reduced pressure to give the desired product. This material was used directly without further purification.

### Intermediate 7

### 8-(4-Methyl-piperazin-1-yl)-5,6,7,8-tetrahydro-naphthalen-2-ol

Intermediate 6 (6.09 mmol) was dissolved in 35 mL of anhydrous acetonitrile and N-methyl piperazine (7.308 mmol) was added, followed by potassium chloride (12.18) and sodium iodide (0.609 mmol). The reaction was heated to 85° C. for 2.5 hours. Upon cooling, the reaction was partitioned between CH₂Cl₂ and H₂O. The organics were extracted and dried over MgSO₄, filtered and concentrated under reduced pressure. Purification was accomplished through flash chromatography on silica gel eluting with a gradient system of 5-10% CH₃OH/CH₂Cl₂. Product containing fractions were combined and concentrated to give the desired product (894 mg, 3.63 mmol, 60% yield); MS 247.3 [M+H].

### Intermediate 8

### Trifluoro-methanesulfonic acid 8-(4-methyl-piperazin-1-yl)-5,6,7,8-tetrahydro-naphthalen-2-yl ester

Intermediate 7 (2.437 mmol) was dissolved in 10 mL of anhydrous THF and cooled to −78° C. NaHMDS (sodium hexamethyldisilazane) (3.655 mmol) was added to the reaction drop wise and the resultant solution was stirred for 0.5 hours. PhNTf₂ (N-phenyl trifluoromethanesulfonimide) (3.04 mmol) was then added and the reaction was stirred at −78° C. for an additional 20 min. The reaction was then allowed to warm to room temperature and was quenched with H₂O. The crude material was then extracted with CH₂Cl₂. Combined organics were dried over MgSO₄, filtered and concentrated under reduced pressure. Purification was accomplished through flash chromatography on silica gel using a gradient system of 100% CH₂Cl₂ ramped to 25% CH₃OH/CH₂Cl₂. Product containing fractions were combined and concentrated under reduced pressure to give the product as a pale oil (790.0 mg, 2.09 mmol, 86% yield); C13 NMR 20.8, 21.8, 29.3, 45.9, 55.7, 62.8, 119.2, 120.6, 130.7, 138.9, 141.0, 148.4.

### Intermediate 9

### 8-(4-Methyl-piperazin-1-yl)-5,6,7,8-tetrahydro-naphthalene-2-carboxylic acid methyl ester

Intermediate 8 (0.53 mmol) was dissolved in 30.0 mL of methanol. Pd(OAc)₂ (0.212 mmol) was added, followed by 1,3-bis(diphenylphosphino)propene (0.14 mmol), triethylamine (0.218 mmol) and 0.85 mL of DMSO. The reaction mixture was then subjected to a CO environment at 50 psi heating at 70° C. for 16 hours. Upon cooling, the reaction was diluted with H₂O and CH₂Cl₂ and filtered through a plug of celite. The organics were extracted and dried over Na₂SO₄, filtered and concentrated under reduced pressure. Purification was accomplished through flash chromatography on silica gel eluting with 4% CH₃OH/CH₂Cl₂. Product containing fractions were combined and concentrated under reduced pressure to give the desired product as a pale oil (59.0 mg, 0.205 mmol, 39% yield); MS 289.3 [M+H].

### Intermediate 10

### 1-Methyl-4-[7-(4,4,5,5-tetramethyl-[1,3,2]dioxaborolan-2-yl)-1,2,3,4-tetrahydro-naphthalen-1-yl]-piperazine

Intermediate 8 (0.53 mmol) was dissolved in 0.6 mL of anhydrous DMF and bis(pinocolato)diboron (1.60 mmol) was added, followed by dichloro[1,1′-bis(diphenylphosphino)ferrocene]palladium(II)dichloromethane adduct (0.027 mmol) and potassium acetate (1.60 mmol). The resultant solution was heated to 80° C. for 4 hours. Upon cooling, the reaction was partitioned between H₂O and CH₂Cl₂. The organics were extracted, dried over Na₂SO₄, filtered and concentrated under reduced pressure to give the desired product (189.0 mg, 0.53 mmol, 100% yield). This material was used directly without further purification.

### Intermediate 11

### 1-(6-Iodo-chroman-4-yl)-4-methyl-piperazine

6-Iodo-chroman-4-ol (1.824 mmol) was dissolved in 20 mL of anhydrous toluene and thionyl chloride (4.56 mmol) was added. The reaction was heated at 40° C. for 1 hour and then cooled and concentrated under reduced pressure. The residual oil was redissolved in 20.0 mL of anhydrous acetonitrile and N-methyl-piperazine (4.56 mmol) was added, followed by potassium carbonate (5.22 mmol) and sodium iodide (0.182 mmol). The resultant solution was then heated to 70° C. for 16 hours. Upon cooling, the reaction was quenched with H₂O and extracted with CH₂Cl₂. Combined organics were dried over MgSO₄, filtered and concentrated under reduced pressure. Purification was accomplished through flash chromatography on silica gel eluting with a gradient system of 100% CH₂Cl₂ to 10% CH₃OH/CH₂Cl₂. Product containing fractions were combined and concentrated under reduced pressure to give the desired product as a pale oil (580.0 mg, 1.62 mmol, 89% yield); MS 359.0 [M+H].

### Intermediate 12

### 1-Methyl-4-[6-(4,4,5,5-tetramethyl-[1,3,2]dioxaborolan-2-yl)-chroman-4-yl]-piperazine

Intermediate 11 (0.20 mmol) was dissolved in 0.25 mL of anhydrous DMF. Bis(pinacolato)diboron (0.22 mmol) was added, followed by potassium carbonate (0.60 mmol) and Pd(dppf)Cl₂.CH₂Cl₂ (0.010 mmol) and the reaction was heated to 80° C. for 4 hours. Upon cooling, the reaction was partitioned between H₂O and CH₂Cl₂. The organics were extracted and dried over Na₂SO₄, filtered and concentrated under reduced pressure to give the desired product. This material was used directly without further purification.

### Intermediate 13

### 4-(7-Hydroxy-1,2,3,4-tetrahydro-naphthalen-1-yl)-piperazine-1-carboxylic acid tert-butyl ester

Intermediate 6 (97.0 mmol) was dissolved in 800 mL of anhydrous acetonitrile and 1-t-butylpiperzine carboxylate (194.0 mmol) was added, followed by potassium carbonate (291.0 mmol) and sodium iodide (97.0 mmol). The resultant solution was heated to 80° C. for 16 hours. Upon cooling, the reaction was concentrated under reduced pressure. Purification was accomplished through flash chromatography on silica gel eluting with 4% CH₃OH/CH₂Cl₂. Product containing fractions were combined and concentrated under reduced pressure to give a pale foam. This material was further purified through flash chromatography on silica gel eluting with 2.5% CH₃OH/CH₂Cl₂. Product containing fractions were combined and concentrated under reduced pressure to give the desired product (6.9 g, 20.8 mmol, 21% yield); ¹³C NMR (CDCl₃, 100 MHz) 21.2, 22.1, 28.6, 29.1, 44.0, 48.4, 53.9, 63.3, 79.6, 113.2, 114.4, 130.5, 133.2, 139.9, 155.1.

### Intermediate 14

### 4-(7-Cyanomethoxy-1,2,3,4-tetrahydro-naphthalen-1-yl)-piperazine-1-carboxylic acid tert-butyl ester

Intermediate 13 (1.5 mmol) was dissolved in 2.5 mL of acetone and potassium carbonate (3.0 mmol) was added, followed by bromoacetonitrile (2.3 mmol). The resultant solution was heated to 50° C. for 22 hours and then stirred at room temperature for an additional 16 hours. The reaction was then diluted in H₂O/CH₂Cl₂. The organics were extracted, dried over Na₂SO₄, filtered and concentrated under reduced pressure. Purification was accomplished through flash chromatography on silica gel eluting with 20% EtOAc/Hexane. Product containing fractions were combined and concentrated under reduced pressure to give the desired product as a colorless oil (26.8 mg, 0.07 mmol, 5% yield); ¹³C NMR (CDCl₃, 100 MHz) 21.2, 22.1, 28.6, 29.1, 44.0, 48.4, 53.9, 63.3, 79.6, 113.2, 114.4, 130.5, 133.2, 139.9, 155.1.

### Intermediate 15

### 4-(7-(2-Amino-ethoxy)-1,2,3,4-tetrahydro-naphthalen-1-yl)-piperazine-1-carboxylic acid tert-butyl ester

Intermediate 14 (0.72 mmol) was dissolved in 1.0 mL of anhydrous ether and cooled to 0° C. in an ice bath. To this solution 0.9 mL of a 1.0 M solution of LAH (Lithium Aluminum Hydride) in Et₂O was added dropwise forming a white precipitate. The resultant suspension was allowed to warm to room temperature and stirred for 30 min. The reaction was then returned to the ice bath and quenched by drop wise addition of 0.3 mL of 1N NaOH. The reaction was warmed to room temperature and diluted with 30.0 mL of THF, dried over Na₂SO₄, and filtered through a plug of celite. The filtrate was concentrated under reduced pressure to give the desired product as a colorless foam. This material was used directly without further purification. MS 376.3 [M+H].

### Intermediate 16

### 4-{7-[2-(5-Fluoro-pyrimidin-2-ylamino)-ethoxy]-1,2,3,4-tetrahydro-naphthalen-1-yl}-piperazine-1-carboxylic acid tert-butyl ester

Intermediate 15 (0.20 mmol) was dissolved in 1.0 mL of anhydrous DMF and 2-chloro-5-fluoro-pyrimidine (0.20 mmol) was added, followed by sodium bicarbonate (0.40 mmol). The resultant solution was heated at 95° C. for 16 hours. Upon cooling, the reaction was partitioned between H₂O and CH₂Cl₂. The organics were extracted and dried over Na₂SO₄, filtered and concentrated under reduced pressure. Purification was accomplished through flash chromatography on silica gel eluting with 2.5% CH₃OH/CH₂Cl₂. Product containing fractions were combined and further purified on a second silica gel column eluting with 25% EtOAc/Hexanes. Product containing fractions were combined and concentrated under reduced pressure to give the desired product as a pale oil (15.0 mg, 0.032 mmol, 16% yield); ¹H NMR (400 MHz. CDCl₃) 1.45 (s, 9H), 1.53-1.67 (m, 2H), 1.84-1.97 (m, 2H), 2.39-2.71 (m, 6H), 3.35-3.50 (m, 4H), 3.72-3.80 (m, 3H), 4.05-4.16 (m, 2H), 5.55 (br s, 1H), 8.71 (dd, 1H, J=2.9, 8.7), 6.95 (d, 1H, J=8.7), 7.30 (d, 1H, J=2.9), 8.7 (s, 2H).

David, we only have proton data . . .

### Intermediate 17

### (5-Fluoro-pyrimidin-2-yl)-[2-(8-piperazin-1-yl-5,6,7,8-tetrahydro-naphthalen-2-yloxy)-ethyl]-amine

Intermediate 16 (0.032 mmol) was dissolved in 0.5 mL of anhydrous CH₂Cl₂ and 0.5 mL of a 2.0 M solution of HCl in Et₂O was added. The reaction was stirred at room temperature for 12 hours and then concentrated under reduced pressure. The solid was then redissolved in saturated aqueous sodium bicarbonate solution and extracted with CH₂Cl₂. The organics were dried over Na₂SO₄, filtered and concentrated under reduced pressure to give the desired product as a colorless oil (7.0 mg, 0.020 mmol, 94% yield); MS 372.2 [M+H].

### Intermediate 18

### 4-[7-(4-Iodo-benzyloxy)-1,2,3,4-tetrahydro-naphthalen-1-yl]-piperazine-1-carboxylic acid tert-butyl ester

Intermediate 13 (1.50 mmol) was dissolved in 20.0 mL of anhydrous acetonitrile and cesium carbonate (1.91 mmol) was added. The resultant suspension was then heated to 60° C. for 15 min and 1-Bromomethyl-4-iodo-benzene (1.91 mmol) was added portion wise. The reaction is heated at 60° C. for an additional 12 hours. Upon cooling, the reaction was partitioned between H₂O and CH₂Cl₂. The organics were extracted and dried over MgSO₄, filtered and concentrated under reduced pressure. Purification was accomplished through flash chromatography on silica gel eluting with 10% EtOAc/Hexanes. Product containing fractions were combined and concentrated under reduced pressure to give the desired product as a colorless solid (563.0 mg, 0.098 mmol, 65% yield); MS 549.0 [M+H].

### General Procedure 1

### Examples 1-3 were Prepared Using General Procedure 1

Intermediate 3, (0.05 mmol) was dissolved in 0.25 mL of anhydrous DMF and added to the starting carboxylic acid (0.075 mmol). HBTU (0.05 mmol) was then added in an additional 0.25 mL of anhydrous DMF, followed by triethylamine (0.125 mmol). The resultant solution was heated to 60° C. for 20 hours. The reaction was then cooled and partitioned between 1.5 mL of 1N NaOH and 2.5 mL of dichloromethane. The organics were extracted and loaded onto an equilibrated SCX-SPE cartridge (conditioned with 5 mL of CH₃OH, followed by two 5 mL washes with CH₂Cl₂). The column was rinsed with 5 mL of CH₃OH and then eluted with 7.5 mL of 1N triethylamine in CH₃OH, collecting 1.25 mL fractions in tared vials. The product containing fractions were dried under a N₂ stream. Purifications were accomplished by HPLC separation on a Waters Symmetry C¹⁸ column (5 mm, 30×150 mm) with a 2.0 mL/min flow rate eluting with a gradient system of 100%, 80%, 0%, (0.1% TFA in H₂O/CH₃CN) injecting each sample in 1.8 mL of DMSO (Table 1).

TABLE 1

| | | | | | | | HPLC | |
|---|---|---|---|---|---|---|---|---|
| | | | | | THEORETICAL | OBSERVED | Retention | |
| EXAMPLE | NAME | A | D | R⁷ | MASS | MS | Time (min.) | |
| 1 | N-{8-[(2- | G² | Phenyl | 4-CF₃ | 417.2 | 418.2 | 7.14 | |
| | Dimethyl- | | | | | | | |
| | amino-ethyl)- | | | | | | | |
| | ethyl-amino]- | | | | | | | |
| | 5,6,7,8- | | | | | | | |
| | tetrahydro- | | | | | | | |
| | naphthalen-2- | | | | | | | |
| | yl}-4- | | | | | | | |
| | trifluoromethyl- | | | | | | | |
| | benzamide | | | | | | | |
| 2 | N-{8-[(2- | G² | Phenyl | 4-F | 423.23 | 424.23 | 6.66 | |
| | Dimethyl- | | | | | | | |
| | amino-ethyl)- | | | | | | | |
| | ethyl-amino]- | | | | | | | |
| | 5,6,7,8- | | | | | | | |
| | tetrahydro- | | | | | | | |
| | naphthalen-2- | | | | | | | |
| | yl}-4-fluoro- | | | | | | | |
| | benzamide | | | | | | | |
| 3 | 4-tert-Butyl-N- | G² | Phenyl | 4-t-butyl | 405.28 | 406.29 | 7.41 | min |
| | {8-[(2- | | | | | | | |
| | dimethylamino- | | | | | | | |
| | ethyl)-ethyl- | | | | | | | |
| | amino]-5,6,7,8- | | | | | | | |
| | tetrahydro- | | | | | | | |
| | naphthalen-2- | | | | | | | |
| | yl}-benzamide | | | | | | | |

### General Procedure 2

### Examples 4-13 were Prepared Using General Procedure 2

Intermediate 8 (0.075 mmol) was dissolved in 0.5 mL of degassed EtOH and added to the corresponding boronic acid having the moieties -DR⁷. Cesium carbonate (0.1875 mmol) was dissolved in degassed H₂O and added to the reaction solution, followed by Pd(Ph₃P)₄ (0.00375 mmol) in 0.18 mL of degassed DME. The resultant mixture was then heated to 90° C. for 19 hours. The reaction was cooled and the crude material partitioned between 1.2 mL of 2 N NaOH and 2.3 mL of CH₂Cl₂. The organics were extracted and then loaded onto an equilibrated 6-mL SCX-SPE cartridge (conditioned with one 5 mL wash with CH₃OH, and two rinses with 5 mL CH₂Cl₂). The products were eluted with 7.5 mL of 1N triethylamine in CH₃OH collecting 1.25 mL fractions. The product containing fractions were dried under a N₂ stream. Purifications were accomplished by HPLC separation on a Waters Symmetry C¹⁸ column (5 mm, 30×150 mm) with a 2.0 mL/min flow rate eluting with a gradient system of 100%, 80%, 0%, (0.1% TFA in H₂O/CH₃CN) injecting each sample in 1.8 mL of DMSO (Table 2).

TABLE 2

| | | | | | | | HPLC |
|---|---|---|---|---|---|---|---|
| | | | | | THEORETICAL | OBSERVED | Retention |
| EXAMPLE | NAME | A | D | R⁷ | MASS | MS | Time (min.) |
| 4 | 1-[7-(4-Benzyl- | — | Phenyl | 4-benzyl | 396.26 | 397.23 | 7.68 |
| | phenyl)-1,2,3,4- | | | | | | |
| | tetrahydro- | | | | | | |
| | naphthalen-1-yl]- | | | | | | |
| | 4-methyl- | | | | | | |
| | piperazine | | | | | | |
| 5 | 1-[7-(4- | — | Phenyl | 4- | 412.25 | 413.25 | 7.44 |
| | Benzyloxy- | | | benzyloxy | | | |
| | phenyl)-1,2,3,4- | | | | | | |
| | tetrahydro- | | | | | | |
| | naphthalen-1-yl]- | | | | | | |
| | 4-methyl- | | | | | | |
| | piperazine | | | | | | |
| 6 | 1-Methyl-4-(7- | — | Phenyl | H | 306.21 | 307.23 | 6.08 |
| | phenyl-1,2,3,4- | | | | | | |
| | tetrahydro- | | | | | | |
| | naphthalen-1-yl)- | | | | | | |
| | piperazine | | | | | | |
| 7 | 1-[7-(4-Fluoro- | — | Phenyl | 4-F | 324.2 | 325.22 | 6.23 |
| | phenyl)-1,2,3,4- | | | | | | |
| | tetrahydro- | | | | | | |
| | naphthalen-1-yl]- | | | | | | |
| | 4-methyl- | | | | | | |
| | piperazine | | | | | | |
| 8 | 1-[7-(3,5- | — | Phenyl | 3,5-Cl | 374.13 | 375.15 | 7.41 |
| | Dichloro- | | | | | | |
| | phenyl)-1,2,3,4- | | | | | | |
| | tetrahydro- | | | | | | |
| | naphthalen-1-yl]- | | | | | | |
| | 4-methyl- | | | | | | |
| | piperazine | | | | | | |
| 9 | 1-[7-(2-Methoxy- | — | Phenyl | 2-CH₃O— | 336.22 | 337.18 | 5.96 |
| | phenyl)-1,2,3,4- | | | | | | |
| | tetrahydro- | | | | | | |
| | naphthalen-1-yl]- | | | | | | |
| | 4-methyl- | | | | | | |
| | piperazine | | | | | | |
| 10 | 1-Methyl-4-[7-(4- | — | Phenyl | 4-CF₃— | 374.2 | 375.17 | 7.08 |
| | trifluoromethyl- | | | | | | |
| | phenyl)-1,2,3,4- | | | | | | |
| | tetrahydro- | | | | | | |
| | naphthalen-1-yl]- | | | | | | |
| | piperazine | | | | | | |
| 11 | 1-[7-(3,4- | — | Phenyl | 3,4-CH₃O— | 366.23 | 367.23 | 5.60 |
| | Dimethoxy- | | | | | | |
| | phenyl)-1,2,3,4- | | | | | | |
| | tetrahydro- | | | | | | |
| | naphthalen-1-yl]- | | | | | | |
| | 4-methyl- | | | | | | |
| | piperazine | | | | | | |
| 12 | 1-(7-Biphenyl-4- | — | Phenyl | 4-Phenyl | 382.24 | 383.24 | 7.39 |
| | yl-1,2,3,4- | | | | | | |
| | tetrahydro- | | | | | | |
| | naphthalen-1-yl)- | | | | | | |
| | 4-methyl- | | | | | | |
| | piperazine | | | | | | |
| 13 | 1-[7-(2,3- | — | Phenyl | ,4- | 364.22 | 365.23 | 5.91 |
| | Dihydro- | | | OCH₂CH₂ | | | |
| | benzo[1,4]dioxin- | | | O— | | | |
| | 6-yl)-1,2,3,4- | | | | | | |
| | tetrahydro- | | | | | | |
| | naphthalen-1-yl]- | | | | | | |
| | 4-methyl- | | | | | | |
| | piperazine | | | | | | |

### General Procedure 3

### Examples 14-32 were Prepared Using General Procedure 3

The starting amine H₂NR² (0.3 mmol) was dissolved in 0.3 mL of anhydrous dichloromethane. To this solution was added 0.125 mL of a 2M solution of AlMe₃ (0.25 mmol) in toluene, and the resultant solution was stirred at room temperature for 30 min. Intermediate 9 (0.05 mmol) was then added in solution with 0.2 mL of anhydrous dichloromethane and the reaction was heated to 50° C. for 19 hours. The reaction was then quenched with 0.1 mL of H₂O (vigorous bubbling evident) and stirred for an additional 20 minutes. The crude material was then partitioned between 1.2 mL of 2 N NaOH and 2.3 mL of CH₂Cl₂. The organics were extracted and loaded onto an equilibrated SCX-SPE cartridge (preconditioned with one 5 mL CH₃OH rinse and two 5 mL washes of CH₂Cl₂). The column was rinsed with 5 mL of CH₃OH and the material eluted with 7.5 mL of 1N triethylamine in CH₃OH, collecting 1.25 mL fractions into tared vials. The product containing fractions were dried under a N₂ stream. Purifications were accomplished by HPLC separation on a Waters Symmetry C¹⁸ column (5 mm, 30×150 mm) with a 2.0 mL/min flow rate eluting with a gradient system of 100%, 80%, 0%, (0.1% TFA in H₂O/CH₃CN) injecting each sample in 1.8 mL of DMSO (Table 3).

TABLE 3

| | | | | | | | HPLC |
|---|---|---|---|---|---|---|---|
| | | | | | THEORETICAL | OBSERVED | Retention |
| EXAMPLE | NAME | A | D | R² | MASS | MS | Time (min.) |
| 14 | 8-(4-Methyl- | G¹ | — | 1-(4-methoxy- | 407.26 | 408.26 | 5.54 |
| | piperazin-1-yl)- | | | phenyl)-ethyl- | | | |
| | 5,6,7,8- | | | | | | |
| | tetrahydro- | | | | | | |
| | naphthalene-2- | | | | | | |
| | carboxylic acid | | | | | | |
| | [1-(4-methoxy- | | | | | | |
| | phenyl)-ethyl]- | | | | | | |
| | amide | | | | | | |
| 15 | 8-(4-Methyl- | G¹ | — | 4-tert-butyl- | 419.29 | 420.32 | 6.64 |
| | piperazin-1-yl)- | | | benzyl- | | | |
| | 5,6,7,8- | | | | | | |
| | tetrahydro- | | | | | | |
| | naphthalene-2- | | | | | | |
| | carboxylic acid | | | | | | |
| | 4-tert-butyl- | | | | | | |
| | benzylamide | | | | | | |
| 16 | 8-(4-Methyl- | G¹ | — | 4- | 431.22 | 432.25 | 6.21 |
| | piperazin-1-yl)- | | | trifluoromethyl- | | | |
| | 5,6,7,8- | | | benzyl | | | |
| | tetrahydro- | | | | | | |
| | naphthalene-2- | | | | | | |
| | carboxylic acid | | | | | | |
| | 4- | | | | | | |
| | trifluoromethyl- | | | | | | |
| | benzylamide | | | | | | |
| 17 | 8-(4-Methyl- | G¹ | — | 1,2,3,4- | 403.26 | 404.29 | 5.88 |
| | piperazin-1-yl)- | | | tetrahydro- | | | |
| | 5,6,7,8- | | | naphthalen-1- | | | |
| | tetrahydro- | | | yl | | | |
| | naphthalene-2- | | | | | | |
| | carboxylic acid | | | | | | |
| | (1,2,3,4- | | | | | | |
| | tetrahydro- | | | | | | |
| | naphthalen-1- | | | | | | |
| | yl)-amide | | | | | | |
| 18 | 8-(4-Methyl- | G¹ | — | 2-(3- | 445.23 | 446.27 | 6.25 |
| | piperazin-1-yl)- | | | trifluoromethyl- | | | |
| | 5,6,7,8- | | | phenyl)-ethyl- | | | |
| | tetrahydro- | | | | | | |
| | naphthalene-2- | | | | | | |
| | carboxylic acid | | | | | | |
| | [2-(3- | | | | | | |
| | trifluoromethyl- | | | | | | |
| | phenyl)-ethyl]- | | | | | | |
| | amide | | | | | | |
| 19 | 8-(4-Methyl- | G¹ | — | 4-chloro- | 397.19 | 398.17 | 5.81 |
| | piperazin-1-yl)- | | | benzyl- | | | |
| | 5,6,7,8- | | | | | | |
| | tetrahydro- | | | | | | |
| | naphthalene-2- | | | | | | |
| | carboxylic acid | | | | | | |
| | 4-chloro- | | | | | | |
| | benzylamide | | | | | | |
| 20 | 8-(4-Methyl- | G¹ | — | 4-fluoro- | 381.22 | 382.22 | 5.38 |
| | piperazin-1-yl)- | | | benzyl- | | | |
| | 5,6,7,8- | | | | | | |
| | tetrahydro- | | | | | | |
| | naphthalene-2- | | | | | | |
| | carboxylic acid | | | | | | |
| | 4-fluoro- | | | | | | |
| | benzylamide | | | | | | |
| 21 | 8-(4-Methyl- | G¹ | — | furan-2-yl- | 353.21 | 354.22 | 4.66 |
| | piperazin-1-yl)- | | | methyl | | | |
| | 5,6,7,8- | | | | | | |
| | tetrahydro- | | | | | | |
| | naphthalene-2- | | | | | | |
| | carboxylic acid | | | | | | |
| | (furan-2- | | | | | | |
| | ylmethyl)- | | | | | | |
| | amide | | | | | | |
| 22 | 8-(4-Methyl- | G¹ | — | 2-chloro- | 397.19 | 398.21 | 5.60 |
| | piperazin-1-yl)- | | | benzyl | | | |
| | 5,6,7,8- | | | | | | |
| | tetrahydro- | | | | | | |
| | naphthalene-2- | | | | | | |
| | carboxylic acid | | | | | | |
| | 2-chloro- | | | | | | |
| | benzylamide | | | | | | |
| 23 | 8-(4-Methyl- | G¹ | — | 3- | 431.22 | 432.24 | 6.17 |
| | piperazin-1-yl)- | | | trifluoromethyl- | | | |
| | 5,6,7,8- | | | benzyl | | | |
| | tetrahydro- | | | | | | |
| | naphthalene-2- | | | | | | |
| | carboxylic acid | | | | | | |
| | 3- | | | | | | |
| | trifluoromethyl- | | | | | | |
| | benzylamide | | | | | | |
| 24 | 8-(4-Methyl- | G¹ | — | 3-fluoro- | 381.22 | 382.25 | 5.41 |
| | piperazin-1-yl)- | | | benzyl | | | |
| | 5,6,7,8- | | | | | | |
| | tetrahydro- | | | | | | |
| | naphthalene-2- | | | | | | |
| | carboxylic acid | | | | | | |
| | 3-fluoro- | | | | | | |
| | benzylamide | | | | | | |
| 25 | 8-(4-Methyl- | G¹ | — | 2-methyl- | 377.25 | 378.28 | 5.51 |
| | piperazin-1-yl)- | | | benzyl | | | |
| | 5,6,7,8- | | | | | | |
| | tetrahydro- | | | | | | |
| | naphthalene-2- | | | | | | |
| | carboxylic acid | | | | | | |
| | 2-methyl- | | | | | | |
| | benzylamide | | | | | | |
| 26 | 8-(4-Methyl- | G¹ | — | 2-methoxy- | 393.24 | 394.28 | 5.35 |
| | piperazin-1-yl)- | | | benzyl | | | |
| | 5,6,7,8- | | | | | | |
| | tetrahydro- | | | | | | |
| | naphthalene-2- | | | | | | |
| | carboxylic acid | | | | | | |
| | 2-methoxy- | | | | | | |
| | benzylamide | | | | | | |
| 27 | 8-(4-Methyl- | G¹ | — | 3-methoxy- | 393.24 | 394.28 | 5.27 |
| | piperazin-1-yl)- | | | benzyl | | | |
| | 5,6,7,8- | | | | | | |
| | tetrahydro- | | | | | | |
| | naphthalene-2- | | | | | | |
| | carboxylic acid | | | | | | |
| | 3-methoxy- | | | | | | |
| | benzylamide | | | | | | |
| 28 | 8-(4-Methyl- | G¹ | — | (2,5-dimethyl- | 367.24 | 368.28 | 4.36 |
| | piperazin-1-yl)- | | | 2H-pyrazol-3- | | | |
| | 5,6,7,8- | | | yl) | | | |
| | tetrahydro- | | | | | | |
| | naphthalene-2- | | | | | | |
| | carboxylic acid | | | | | | |
| | (2,5-dimethyl- | | | | | | |
| | 2H-pyrazol-3- | | | | | | |
| | yl)-amide | | | | | | |
| 29 | 8-(4-Methyl- | G¹ | — | (5-tert-butyl-2- | 409.28 | 410.32 | 5.39 |
| | piperazin-1-yl)- | | | methyl-2H- | | | |
| | 5,6,7,8- | | | pyrazol-3-yl)- | | | |
| | tetrahydro- | | | | | | |
| | naphthalene-2- | | | | | | |
| | carboxylic acid | | | | | | |
| | (5-tert-butyl-2- | | | | | | |
| | methyl-2H- | | | | | | |
| | pyrazol-3-yl)- | | | | | | |
| | amide | | | | | | |
| 30 | 8-(4-Methyl- | G¹ | — | [1-(4-chloro- | 411.21 | 412.26 | 6.00 |
| | piperazin-1-yl)- | | | phenyl)-ethyl] | | | |
| | 5,6,7,8- | | | | | | |
| | tetrahydro- | | | | | | |
| | naphthalene-2- | | | | | | |
| | carboxylic acid | | | | | | |
| | [1-(4-chloro- | | | | | | |
| | phenyl)-ethyl]- | | | | | | |
| | amide | | | | | | |
| 31 | 8-(4-Methyl- | G¹ | — | 3-chloro- | 397.19 | 398.24 | 5.79 |
| | piperazin-1-yl)- | | | benzyl | | | |
| | 5,6,7,8- | | | | | | |
| | tetrahydro- | | | | | | |
| | naphthalene-2- | | | | | | |
| | carboxylic acid | | | | | | |
| | 3-chloro- | | | | | | |
| | benzylamide | | | | | | |
| 32 | 8-(4-Methyl- | G¹ | — | (pyridin-2- | 364.23 | 365.28 | 3.58 |
| | piperazin-1-yl)- | | | ylmethyl)- | | | |
| | 5,6,7,8- | | | | | | |
| | tetrahydro- | | | | | | |
| | naphthalene-2- | | | | | | |
| | carboxylic acid | | | | | | |
| | (pyridin-2- | | | | | | |
| | ylmethyl)- | | | | | | |
| | amide | | | | | | |

### General Procedure 4

### Examples 33-37 were Prepared Using General Procedure 4

The starting amine H₂NDR² (0.3 mmol) was dissolved in 0.3 mL of anhydrous dichloromethane. To this solution was added 0.125 mL of a 2M solution of AlMe₃ (0.25 mmol) in toluene, and the resultant solution was stirred at room temperature for 30 min. Intermediate 9 (0.05 mmol) was then added in solution with 0.2 mL of anhydrous dichloromethane and the reaction was heated to 50° C. for 19 hours. The reaction was quenched by adding 1.0 mL H₂O and then stirred an additional 20 min. The reaction mixture was then partitioned between 1.0 mL of 2 N NaOH and 2.0 mL of CH₂Cl₂ and the organics were extracted. The crude material was then loaded onto an equilibrated SCX-SPE cartridge (preconditioned with one 5.0 mL rinse of CH₃OH and two 5.0 mL rinses with CH₂Cl₂). The column was rinsed with one 2.0 mL portion of CH₃OH and then eluted with 7.5 mL of 1N triethylamine in CH₃OH, collecting 1.25 mL fractions into tared vials. The product containing fractions were dried under a N₂ stream. Purifications were accomplished by HPLC separation on a Waters Symmetry C¹⁸ column (5 mm, 30×150 mm) with a 2.0 mL/min flow rate eluting with a gradient system of 100%, 80%, 0%, (0.1% TFA in H₂O/CH₃CN) injecting each sample in 0.9 mL of DMSO (Table 4).

TABLE 4

| | | | | | | | | HPLC |
|---|---|---|---|---|---|---|---|---|
| | | | | | | THEORETICAL | OBSERVED | Retention |
| EXAMPLE | NAME | A | D | R² | [symbol] | MASS | MS | Time (min.) |
| 33 | 8-(4-Methyl- | G¹ | Phenyl | | 4-Cl | 383.18 | 384.23 | 6.10 |
| | piperazin-1-yl)- | | | | | | | |
| | 5,6,7,8- | | | | | | | |
| | tetrahydro- | | | | | | | |
| | naphthalene-2- | | | | | | | |
| | carboxylic | | | | | | | |
| | acid(4-chloro- | | | | | | | |
| | phenyl)-amide | | | | | | | |
| 34 | 8-(4-Methyl- | G¹ | — | (5- | — | 371.18 | 372.24 | 4.88 |
| | piperazin-1-yl)- | | | methyl- | | | | |
| | 5,6,7,8- | | | [1,3,4]thiadiazol- | | | | |
| | tetrahydro- | | | 2- | | | | |
| | naphthalene-2- | | | yl) | | | | |
| | carboxylic | | | | | | | |
| | acid(5-methyl- | | | | | | | |
| | [1,3,4]thiadiazol- | | | | | | | |
| | 2-yl)-amide | | | | | | | |
| 35 | 8-(4-Methyl- | G¹ | — | 3,4- | — | 399.21 | 400.27 | 5.63 |
| | piperazin-1-yl)- | | | difluoro- | | | | |
| | 5,6,7,8- | | | benzyl | | | | |
| | tetrahydro- | | | | | | | |
| | naphthalene-2- | | | | | | | |
| | carboxylic | | | | | | | |
| | acid3,4- | | | | | | | |
| | difluoro- | | | | | | | |
| | benzylamide | | | | | | | |
| 36 | 8-(4-Methyl- | G¹ | — | 4- | — | 393.24 | 394.28 | 5.24 |
| | piperazin-1-yl)- | | | methoxy- | | | | |
| | 5,6,7,8- | | | benzyl | | | | |
| | tetrahydro- | | | | | | | |
| | naphthalene-2- | | | | | | | |
| | carboxylic | | | | | | | |
| | acid4-methoxy- | | | | | | | |
| | benzylamide | | | | | | | |
| 37 | 8-(4-Methyl- | G¹ | Pyridin- | H | — | 350.21 | 351.28 | 4.09 |
| | piperazin-1-yl)- | | 2-yl | | | | | |
| | 5,6,7,8- | | | | | | | |
| | tetrahydro- | | | | | | | |
| | naphthalene-2- | | | | | | | |
| | carboxylic acid | | | | | | | |
| | pyridin-2- | | | | | | | |
| | ylamide | | | | | | | |

### General Procedure 5

### Examples 38-40 were Prepared Using General Procedure 5

The starting boronic acid (HO)₂BDR⁷ (0.05 mmol) was combined with intermediate 8 (0.025 mmol) in 0.48 mL of degassed anhydrous THF. K₃PO₄ (0.0625 mL) was then added in 0.06 mL of degassed H₂O, followed by Pd(Ph₃P)₄ in 0.06 mL of degassed DME and the resultant solution was heated at 90° C. for 19 hours. The crude reaction mixture was then cooled and partitioned between 1.0 mL of 1N NaOH and 2.0 mL CH₂Cl₂. The organics were extracted and loaded on an equilibrated 6 mL SCX-SPE cartridge (preconditioned with one 5.0 mL CH₃OH rinse and two 5.0 mL rinses of CH₂Cl₂). The column was rinsed with 5.0 mL of CH₃OH and then eluted with 7.5 mL of 1N triethylamine in CH₃OH, collecting 1.25 mL fractions into tared vials. The product containing fractions were dried under a N₂ stream. Purifications were accomplished by HPLC separation on a Waters Symmetry C⁸ column (5 mm, 30×150 mm) with a 2.0 mL/min flow rate eluting with a gradient system of 100%, 80%, 0%, (0.1% TFA in H₂O/CH₃CN) injecting each sample in 0.9 mL of DMSO (Table 5).

TABLE 5

| | | | | | THEORETICAL | OBSERVED | HPLC Retention |
|---|---|---|---|---|---|---|---|
| EXAMPLE | NAME | A | D | R⁷ | MASS | MS | Time (min.) |
| 38 | 1-Methyl-4-(7-o- | — | Phenyl | 2-CH₃— | 320.23 | 321.27 | 6.46 |
| | tolyl-1,2,3,4- | | | | | | |
| | tetrahydro- | | | | | | |
| | naphthalen-1-yl)- | | | | | | |
| | piperazine | | | | | | |
| 39 | 1-[7-(3,4-Dichloro- | — | Phenyl | 3,4-Cl— | 374.13 | 375.18 | 7.23 |
| | phenyl)-1,2,3,4- | | | | | | |
| | tetrahydro- | | | | | | |
| | naphthalen-1-yl]- | | | | | | |
| | 4-methyl- | | | | | | |
| | piperazine | | | | | | |
| 40 | 1-[7-(3-Methoxy- | — | Phenyl | 3- | 336.22 | 337.27 | 6.06 |
| | phenyl)-1,2,3,4- | | | CH₃O— | | | |
| | tetrahydro- | | | | | | |
| | naphthalen-1-yl]- | | | | | | |
| | 4-methyl- | | | | | | |
| | piperazine | | | | | | |

### General Procedure 6

### Examples 41-48 were Prepared Using General Procedure 6

The starting acid R⁷DCO₂H (0.336 mmol) was dissolved in 2.0 mL of anhydrous DMF in a sealed vial. CDI (0.336 mmol) was added and the reaction was heated to 90° C. for 1 hour. Intermediate 3 (0.305) was then added and the reaction was stirred at 115° C. for an additional 48 hours. Upon cooling, the reaction solution was diluted with 2.0 mL of CH₂Cl₂ and washed with 1.0 mL of 1N NaOH. The organics were extracted and concentrated under reduced pressure. The crude material was then purified through flash chromatography eluting with a gradient system of 5% to 10% to 15% CH₃OH/CH₂Cl₂, collecting 13 mm fractions. Product containing fractions were then combined and concentrated under reduced pressure to give the desired analogs (Table 6).

TABLE 6

| | | | | | THEORETICAL | OBSERVED |
|---|---|---|---|---|---|---|
| EXAMPLE | NAME | A | D | R⁷ | MASS | MS |
| 41 | 4-tert-Butyl-N-[8- | G² | Phenyl | 4-tert-Butyl | 405.6 | 406.3 |
| | (4-methyl- | | | | | |
| | piperazin-1-yl)- | | | | | |
| | 5,6,7,8- | | | | | |
| | tetrahydro- | | | | | |
| | naphthalen-2-yl]- | | | | | |
| | benzamide | | | | | |
| 42 | 2-Methoxy-N-[8- | G² | Phenyl | 2-CH₃O—, | 464.6 | 465.3 |
| | (4-methyl- | | | 4-morpholin- | | |
| | piperazin-1-yl)- | | | 4-yl | | |
| | 5,6,7,8- | | | | | |
| | tetrahydro- | | | | | |
| | naphthalen-2-yl]- | | | | | |
| | 4-morpholin-4-yl- | | | | | |
| | benzamide | | | | | |
| 43 | 4-Isopropoxy-N- | G² | Phenyl | 4-Isopropoxy | 407.6 | 408.2 |
| | [8-(4-methyl- | | | | | |
| | piperazin-1-yl)- | | | | | |
| | 5,6,7,8- | | | | | |
| | tetrahydro- | | | | | |
| | naphthalen-2-yl]- | | | | | |
| | benzamide | | | | | |
| 44 | 4-Benzyloxy-N-[8- | G² | Phenyl | 4-Benzyloxy- | 455.6 | 456.2 |
| | (4-methyl- | | | | | |
| | piperazin-1-yl)- | | | | | |
| | 5,6,7,8- | | | | | |
| | tetrahydro- | | | | | |
| | naphthalen-2-yl]- | | | | | |
| | benzamide | | | | | |
| 45 | Benzo[1,3]dioxole- | G² | Phenyl | 3,4-OCH₂O— | 393.5 | 394.2 |
| | 5-carboxylic acid | | | | | |
| | [8-(4-methyl- | | | | | |
| | piperazin-1-yl)- | | | | | |
| | 5,6,7,8- | | | | | |
| | tetrahydro- | | | | | |
| | naphthalen-2-yl]- | | | | | |
| | amide | | | | | |
| 46 | 4-(Cyclohex-1- | G² | Phenyl | 4-(Cyclohex- | 445.6 | 446.2 |
| | enyloxy)-N-[8-(4- | | | 1-enyloxy) | | |
| | methyl-piperazin- | | | | | |
| | 1-yl)-5,6,7,8- | | | | | |
| | tetrahydro- | | | | | |
| | naphthalen-2-yl]- | | | | | |
| | benzamide | | | | | |
| 47 | N-{8-[(2- | G² | Phenyl | 2-F, | 452.6 | 453.2 |
| | Dimethylamino- | | | 4- | | |
| | ethyl)-methyl- | | | morpholinyl | | |
| | amino]-5,6,7,8- | | | | | |
| | tetrahydro- | | | | | |
| | naphthalen-2-yl}- | | | | | |
| | 2-fluoro-4- | | | | | |
| | morpholin-4-yl- | | | | | |
| | benzamide | | | | | |
| 48 | N-[8-(4-Methyl- | G² | Phenyl | 4-CF₃O— | 433.5 | 434.1 |
| | piperazin-1-yl)- | | | | | |
| | 5,6,7,8- | | | | | |
| | tetrahydro- | | | | | |
| | naphthalen-2-yl]- | | | | | |
| | 4- | | | | | |
| | trifluoromethoxy- | | | | | |
| | benzamide | | | | | |

### General Procedure 7

### Examples 49-53 were Prepared Using General Procedure 7

Pd(dppf)Cl₂.CH₂Cl₂ catalyst (0.027 mmol), the pyridine R⁷-Hal (0.643 mmol), and sodium carbonate (3.8 mmol) were combined in 1.8 mL of water. Intermediate 10 (0.53 mmol) was then added, followed by 0.5 mL of DMF and the resultant solution was heated to 80° C. for 16 hours. Upon cooling, the reaction was partitioned between 2.0 mL H₂O and 4.0 mL CH₂Cl₂. The organics were extracted and dried over Na₂SO₄, filtered and concentrated under reduced pressure. The crude material was then purified through flash chromatography, eluting with a gradient system of 5-10% CH₃OH/CH₂Cl₂, collecting 13 mm fractions. Product containing fractions were combined and concentrated under reduced pressure to give the desired analogs (Table 7).

TABLE 7

| | | | | | THEORETICAL | OBSERVED |
|---|---|---|---|---|---|---|
| EXAMPLE | NAME | A | D | R⁷ | MASS | MS |
| 49 | 1-Methyl-4-(7- | — | pyridin-4-yl- | H | 307.4 | 308.3 |
| | pyridin-4-yl-1,2,3,4- | | | | | |
| | tetrahydro- | | | | | |
| | naphthalen-1-yl)- | | | | | |
| | piperazine | | | | | |
| 50 | 1-Methyl-4-[7-(4- | — | pyridin-3-yl | 4-CH₃— | 321.5 | 322.3 |
| | methyl-pyridin-3-yl)- | | | | | |
| | 1,2,3,4-tetrahydro- | | | | | |
| | naphthalen-1-yl]- | | | | | |
| | piperazine | | | | | |
| 51 | 1-Methyl-4-[7-(6- | — | pyridin-3-yl | 6-CH₃— | 321.5 | 322.2 |
| | methyl-pyridin-3-yl)- | | | | | |
| | 1,2,3,4-tetrahydro- | | | | | |
| | naphthalen-1-yl]- | | | | | |
| | piperazine | | | | | |
| 52 | 1-[7-(6-Methoxy- | — | pyridin-3-yl | 6-CH₃O— | 337.5 | 338.2 |
| | pyridin-3-yl)-1,2,3,4- | | | | | |
| | tetrahydro- | | | | | |
| | naphthalen-1-yl]-4- | | | | | |
| | methyl-piperazine | | | | | |
| 53 | 1-Methyl-4-(7- | — | 7-pyridin-2- | H | 307.4 | 308.2 |
| | pyridin-2-yl-1,2,3,4- | | yl | | | |
| | tetrahydro- | | | | | |
| | naphthalen-1-yl)- | | | | | |
| | piperazine | | | | | |
| 54 | 4-{5-[8-(4-Methyl- | — | 5-pyridin-2- | 2- | 392.3 | 393.2 |
| | piperazin-1-yl)- | | yl | morpholin- | | |
| | 5,6,7,8-tetrahydro- | | | 4-yl | | |
| | naphthalen-2-yl]- | | | | | |
| | pyridin-2-yl}- | | | | | |
| | morpholine | | | | | |

### EXAMPLES 55-56

The enantiomers of example 50 were isolated on a Chiralcel OD column (5 cm×50 cm) with a 70 mL/min flow rate eluting with a 90/10 Heptane/Isopropanol system.

### EXAMPLES 57-61

Examples 57-61 (TABLE 8) were prepared according to General Procedure 7 using intermediate 12.

TABLE 8

| | | | | | | THEORETICAL | OBSERVED |
|---|---|---|---|---|---|---|---|
| EXAMPLE | NAME | A | D | R² | R⁷ | MASS | MS |
| 57 | 1-Methyl-4- | — | pyridin-3-yl | — | 4-CH₃— | 323.4 | 324.2 |
| | [6-(4- | | | | | | |
| | methyl- | | | | | | |
| | pyridin-3- | | | | | | |
| | yl)- | | | | | | |
| | chroman-4- | | | | | | |
| | yl]- | | | | | | |
| | piperazine | | | | | | |
| 58 | 1-Methyl-4- | — | pyridin-4- | — | H | 309.4 | 310.2 |
| | (6-pyridin- | | yl- | | | | |
| | 4-yl- | | | | | | |
| | chroman-4- | | | | | | |
| | yl)- | | | | | | |
| | piperazine | | | | | | |
| 59 | 4-{5-[4-(4- | — | pyridin-3-yl | — | 5-morpholin- | 394.5 | 395.2 |
| | Methyl- | | | | 4-yl | | |
| | piperazin-1- | | | | | | |
| | yl)- | | | | | | |
| | chroman-6- | | | | | | |
| | yl]-pyridin- | | | | | | |
| | 3-yl}- | | | | | | |
| | morpholine | | | | | | |

### General Procedure 8

### EXAMPLE 62

### 1-Methyl-4-[7-(4-trifluoromethyl-benzyloxy)-1,2,3,4-tetrahydro-naphthalen-1-yl]-piperazine

The title compound was prepared according to the following procedure (General Procedure 8): 4-[7-(4-Trifluoromethyl-benzyloxy)-1,2,3,4-tetrahydro-naphthalen-1-yl]-piperazine-1-carboxylic acid tert-butyl ester (0.106 mmol) was dissolved in 5 mL of anhydrous THF. To this solution was added 0.4 mL of a 1M solution of LAH/THF. The reaction mixture was heated at 55° C. for 48 hours. The reaction was then quenched with 2 mL of 1N Nab H, stirred for 30 minutes and then extracted with 5 mL of CH₂Cl₂. The combined organics were then dried over MgSO₄, filtered and concentrated under reduced pressure. The crude material purified through flash chromatography on silica gel, eluting with 5% CH₃OH/CH₂Cl₂. Product containing fractions were combined and concentrated under reduced pressure to give the desired product (21.0 mg, 0.052 mmol, 49% yield); MS 405.6 [M+H].

### EXAMPLE 63

### 1-[7-(4-tert-Butyl-benzyloxy)-1, 2, 3, 4-tetrahydro-naphthalen-1-yl]-4-methyl-piperazine

The title compound was prepared according to General Procedure 8 using 4-[7-(4-tert-Butyl-benzyloxy)-1,2,3,4-tetrahydro-naphthalen-1-yl]-piperazine-1-carboxylic acid tert-butyl ester to give the desired product (6.0 mg, 0.015 mmol, 14% yield); MS 393.5 [M+H].

### General Procedure 9

### EXAMPLE 64

### 6-Morpholin-4-yl-nicotinic acid 8-(4-methyl-piperazin-1-yl)-5,6,7,8-tetrahydro-naphthalen-2-yl ester

8-(4-Methyl-piperazin-1-yl)-5,6,7,8-tetrahydro-naphthalen-2-ol (1.02 mmol), DCC (1.18 mmol), 6-Morpholin-4-yl-nicotinic acid (1.12 mmol) and DMAP (0.2 mmol) were all combined in 10.0 mL of anhydrous CH₂Cl₂ and stirred at room temperature for 16 hours. The reaction was then partitioned with 20.0 mL of fresh CH₂Cl₂ and 40.0 mL of H₂O. The organics were extracted and then dried over MgSO₄, filtered and concentrated under reduced pressure. Purification was accomplished through flash chromatography on silica gel eluting with a gradient system of 100% CH₂Cl₂ to 5% CH₃OH/CH₂Cl₂. The product containing fractions were combined and concentrated under reduced pressure to give the desired material as colorless solid (209.9 mg, 0.481 mmol, 47% yield); MS 437.1 [M+H].

### General Procedure 10

### EXAMPLE 65

### N-[8-(4-Methyl-piperazin-1-yl)-5,6,7,8-tetrahydro-naphthalen-2-yl]-6-morpholin-4-yl-nicotinamide

To 6-Morpholin-4-yl-nicotinic acid dissolved in 5.0 mL of anhydrous DMF was added carbodiimidazole (CDI), (0.672 mmol) and heated to 90° C. for 1 hour. 8-(4-Methyl-piperazin-1-yl)-5,6,7,8-tetrahydro-naphthalen-2-ylamine (0.56 mmol) was then added and the reaction mixture was further heated to 110° C. for 16 hours. The crude material was then concentrated under a N₂ stream and purified by flash chromatography on silica gel, eluting with a gradient system of 5%-10% CH₃OH/CH₂Cl₂. Product containing fractions were then combined and concentrated under reduced pressure to give the desired material as a yellow solid. This product was further purified through crystallization with CH₃Cl/Hexanes to give the clean racemic product as a colorless solid; MS 436.3 [M+H]. The enantiomers were separated on a Chiralpak AD column (4.6 mm×25 cm) with a 1.0 mL/min flow rate eluting with 50% Heptane/Isopropanol system.

### EXAMPLE 66

### {4-[8-(4-Methyl-piperazin-1-yl)-5,6,7,8-tetrahydro-naphthalen-2-ylcarbamoyl]-benzyl}-carbamic acid tert-butyl ester

The title compound was prepared following the procedure detailed in General Procedure 9, using 4-(tert-Butoxycarbonylamino-methyl)benzoic acid to obtain the desired product as a colorless solid (80.0 mg, 0.017 mmol, 54% yield); MS 479.2 [M+H].

### General Procedure 11

### EXAMPLE 67

### N-[8-(4-Methyl-piperazin-1-yl)-5,6,7,8-tetrahydro-naphthalen-2-yl]-4-trifluoromethyl-benzamide

Trifluoromethanesulfonic acid 8-(4-methyl-piperazin-1-yl)-5,6,7,8-tetrahydro-naphthalen-2-yl ester (0.211 mmol), Pd₂(dba)₃ (0.011 mmol), Xantphos (0.032 mmol), cesium carbonate (0.296 mmol) and 4-trifluoromethyl-benzamide (0.253 mmol) were all combined in 2.0 mL of anhydrous dioxane and heated to 100° C. for 10 hours. Upon cooling, the reaction was partitioned with 5.0 mL CH₂Cl₂ and 10 mL of H₂O. The organics were extracted and then dried over MgSO₄, filtered and concentrated under reduced pressure. Purification was accomplished through flash chromatography on silica gel eluting with a gradient system of 100% CH₂Cl₂ to 10% CH₃OH/CH₂Cl₂. Product containing fractions were combined and concentrated under reduced pressure to give the desired material; MS 418.6 [M+H].

### EXAMPLE 68

### 2-[8-(4-Methyl-piperazin-1-yl)-5,6,7,8-tetrahydro-naphthalen-2-yl]-6-morpholin-4-yl-3,4-dihydro-2H-isoquinolin-1-one

The title compound was prepared according to General Procedure 11, using intermediate 8 and 6-morpholin-4-yl-3,4-dihydro-2H-isoquinolin-1-one to give the desired product (5.0 mg, 0.001 mmol, 23% yield); MS 461.3 [M+H]. Diagnostic ¹³C NMR (100 MHz, CDCl₃) 21.7, 22.0, 29.5, 48.2, 49.7, 55.7, 62.9, 66.9, 112.0, 113.3, 121.1, 123.7, 124.5, 129.5, 130.5, 136.4, 140.2, 141.4, 153.8, 164.6, 184.9.

### General Procedure 12

### EXAMPLE 69

### 1-Methyl-4-(7-piperidin-4-yl-1,2,3,4-tetrahydro-naphthalen-1-yl)-piperazine

The title compound was prepared according to the following procedure (General Procedure 12): 1-Methyl-4-(7-pyridin-4-yl-1,2,3,4-tetrahydro-naphthalen-1-yl)piperazine (0.19 mmol) was dissolved in 1.0 mL of acetic acid. PtO₂ (20.0 mg, 35% by weight) was added and the reaction was subjected to hydrogenation at 40 psi for 1.5 hours. A second 20.0 mg portion of PtO₂ was then added and the reaction returned to a 40 psi hydrogenation atmosphere for an additional 1.5 hours. The crude material was then diluted with 10 mL EtOH and filtered through a plug of celite. The solution was then basified with saturated aqueous sodium bicarbonate solution to pH 8.0 and triturated with CH₂Cl₂. The suspension was then filtered through celite and rinsed with CH₂Cl₂. The organics were then concentrated to give the desired product as a colorless oil (37.0 mg, 0.118 mmol, 62% yield); MS 314.3 [M+H].

### EXAMPLE 70

### 1-Methyl-4-(7-piperidin-3-yl-1,2,3,4-tetrahydro-naphthalen-1-yl)-piperazine

The title compound was prepared according to General Procedure 12, using 1-Methyl-4-(7-pyridin-3-yl-1,2,3,4-tetrahydro-naphthalen-1-yl)-piperazine to give the desired product (160.0 mg, 0.510 mmol, 64% yield); MS 314.1 [M+H].

### General Procedure 13

### EXAMPLE 71

### 1-Methyl-4-[7-(1-methyl-piperidin-4-yl)-1,2,3,4-tetrahydro-naphthalen-1-yl]-piperazine

1-Methyl-4-(7-piperidin-4-yl-1,2,3,4-tetrahydro-naphthalen-1-yl-piperazine (0.054 mmol) was dissolved in 0.23 mL of THF. Formic acid (0.11 mmol) was added, followed by 37% aqueous formalin (0.065 mmol) in 0.05 mL of H₂O. The resultant solution was heated to 80° C. for 3 h and then cooled to room temperature overnight. The reaction was partitioned between 5.0 mL of saturated aqueous sodium bicarbonate solution and 5.0 mL of CH₂Cl₂. The organics were extracted and then dried over Na₂SO₄, filtered and concentrated under reduced pressure. Purification was accomplished through flash chromatography on silica gel eluting with 8% CH₃OH/CH₂Cl₂ with 0.5% NH₄OH. Product containing fractions were combined and concentrated under reduced pressure to give the desired material as a colorless oil (7.0 mg, 0.021 mmol, 19% yield); MS 328.3 [M+H].

### EXAMPLE 72

### (5-Fluoro-pyrimidin-2-yl)-{2-[8-(4-methyl-piperazin-1-yl)-5,6,7,8-tetrahydro-naphthalen-2-yloxy]-ethyl}-amine

The title compound was prepared according to General Procedure 13, using (5-Fluoro-pyrimidin-2-yl)[2-(8-piperazin-1-yl-5,6,7,8-tetrahydro-naphthalen-2-loxy-ethyl]-amine to obtain the desired product (5.0 mg, 0.0013 mmol, 68% yield); MS 386.2 [M+H].

### EXAMPLE 73

### N-{2-[8-(4-Methyl-piperazin-1-yl)-5,6,7,8-tetrahydro-naphthalen-2-yloxy]-ethyl}-4-trifluoromethyl-benzamide

The title compound was prepared according to General Procedure 13, using N-[2-(8-piperazin-1-yl-5,6,7,8-tetrahydro-naphthalen-2-yloxy)-ethyl]-4-trifluoromethyl-benzamide to give the desired product as a colorless solid (14.0 mg, 0.003 mmol, 37% yield); MS 462.2 [M+H].

### EXAMPLE 74

### 1-Methyl-4-[7-(1-methyl-piperidin-3-yl)-1,2,3,4-tetrahydro-naphthalen-1-yl]-piperazine

The title compound was prepared according to General Procedure 13, using 1-Methyl-4-(7-piperidin-3-yl-1,2,3,4-tetrahydro-naphthalen-1-yl)-piperazine to give the desired product as a colorless solid (5.0 mg, 0.0015 mmol, 24% yield); MS328.3 [M+H].

### General Procedure 14

### EXAMPLE 75

### {4-[8-(4-Methyl-piperazin-1-yl)-5,6,7,8-tetrahydro-naphthalen-2-yl]-piperidin-1-yl}-(4-trifluoromethyl-phenyl)-methanone

1-Methyl-4-(7-piperidin-4-yl-1,2,3,4-tetrahydro-naphthalen-1-yl)piperazine (0.096 mmol), HBTU (0.10 mmol), triethylamine (0.29 mmol) and p-trifluoromethylbenzoic acid (0.19 mmol) were all combined in 1.2 mL of CH₂Cl₂ and 0.5 mL DMF. The resultant solution was heated to 60° C. for 4 hours. Upon cooling, the reaction was partitioned between 5.0 mL CH₂Cl₂ and 5.0 mL 1N NaOH. The organics were extracted and then dried over Na₂SO₄, filtered and concentrated under reduced pressure. Purification was accomplished through flash chromatography on silica gel using a gradient system of 5-8% CH₃OH/CH₂Cl₂. Product containing fractions were combined and concentrated under reduced pressure to give the desired material as a colorless oil (24.0 mg, 0.049 mmol, 51% yield); MS 486.3 [M+H].

### EXAMPLE 76

### {3-[8-(4-Methyl-piperazin-1-yl)-5,6,7,8-tetrahydro-naphthalen-2-yl-piperidin-1-yl}-4-trifluoromethyl-phenyl)-methanone

The title compound was prepared according to General Procedure 14, using 1-Methyl-4-(7-piperidin-3-yl-1,2,3,4-tetrahydro-naphthalen-1-yl)piperazine to give the desired product as a pale solid (14.0 mg, 0.0029 mmol, 13% yield); MS 486.1 [M+H].

### General Procedure 15

### EXAMPLE 77

### 4-Aminomethyl-N-[8-(4-methyl-piperazin-1-yl)-5,6,7,8-tetrahydro-naphthalen-2-yl]-benzamide

{4-[8-(4-Methyl-piperazin-1-yl)-5,6,7,8-tetrahydro-naphthalen-2-ylcarbamoyl]-benzyl}carbamic acid tert-butyl ester (0.167 mmol) was dissolved in 2.0 mL of dichloromethane and 5.0 mL of a 2N HCl solution in Et₂O was added. The reaction was stirred at room temperature for 16 hours and then quenched with 5.0 mL of saturated aqueous sodium bicarbonate solution. The organics were extracted and then dried over MgSO₄, filtered and concentrated under reduced pressure to give the desired product as a colorless solid (63.0 mg, 0.166 mmol, 100% yield); MS 379 [M+H].

### General Procedure 16

### EXAMPLE 78

### (+) and (−) enantiomers of 1-Methyl-4-(7-pyridin-4-yl-1,2,3,4-tetrahydro-naphthalen-1-yl)-piperazine

The separate enantiomers of 1-Methyl-4-(7-pyridin-4-yl-1,2,3,4-tetrahydro-naphthalen-1-yl)-piperazine were isolated on a Chiralpak AD column (10 cm×50 cm) with a 275 mL/min flow rate eluting with 90/10 Heptane/Isopropanol.

### General Procedure 17

### EXAMPLE 79

### 4-(1-Hydroxy-1-methyl-ethyl)-N-[8-(4-methyl-piperazin-1-yl)-5,6,7,8-tetrahydro-naphthalen-2-yl]-benzamide

N-[8-(4-Methyl-piperazin-1-yl)-5,6,7,8-tetrahydro-naphthalen-2-yl]-terephthalamic acid methyl ester (1.5 mmol) was dissolved in 1.0 mL of anhydrous THF and cooled to −78° C. in a dry ice/acetonitrile bath. MeMgBr (14.8 mmol) was added and the reaction was warmed to 0° C. in an ice water bath for 1.5 hours. The reaction was quenched with the slow addition of H₂O and the resultant solution was poured into a mixture of saturated aqueous sodium bicarbonate solution and dichloromethane. The organics were extracted and then dried over Na₂SO₄, filtered and concentrated under reduced pressure. Purification was accomplished through flash chromatography on silica gel eluting with 10% CH₃OH/CH₂Cl₂/0.5% NH₄OH. Product containing fractions were combined and concentrated under reduced pressure to give the desired product as a colorless oil. The HCl salt was formed by dissolving the product (0.039 mmol) in CH₂Cl₂ and adding 2M HCl solution in Et₂O. The desired material was isolated through filtration as a colorless solid (14.0 mg, 0.032 mmol, 21% yield); MS 409.1 [M+H].

### General Procedure 18

### EXAMPLE 80

### 3-[8-(4-Methyl-piperazin-1-yl)-5,6,7,8-tetrahydro-naphthalen-2-yl]-6′-morpholin-4-yl-3,4,5,6-tetrahydro-2H-[1,2′]bipyridinyl

1-Methyl-4-(7-piperidin-3-yl-1,2,3,4-tetrahydro-naphthalen-1-yl)piperazine (0.16 mmol) and 4-(6-Bromo-pyridin-2-yl)morpholine (0.16 mmol) were dissolved in 0.5 mL of anhydrous toluene. Pd(OAc)₂ (0.0032 mmol) was added, followed by racemic BINAP (0.0032 mmol). The resultant solution was heated to 100° C. for 16 hr. Upon cooling, the reaction was poured into a mixture of diluted aqueous sodium bicarbonate solution and dichloromethane. The organics were extracted and dried over Na₂SO₄, filtered and concentrated under reduced pressure. Purification was accomplished through flash chromatography on silica gel eluting with 7% CH₃OH/CH₂Cl₂. Product containing fractions were combined and concentrated under reduced pressure to give the desired product (12.0 mg, 0.0025 mmol, 16% yield); MS ¹³C NMR (100 MHz, CDCl₃) (mixture of diastereomers) 21.8, 22.0, 25.3, 29.6, 32.6, 42.1, 42.2, 45.9, 46.0, 46.1, 52.8, 53.0, 55.8, 63.0, 67.1, 95.1, 95.2, 96.9, 112.5, 125.4, 125.7, 126.6, 126.9, 129.2, 136.8, 139.4, 142.1, 158.4, 158.9. MS 476.4 [M+H].

### Preparation 19

### EXAMPLE 81

### 4-[4-(8-piperazin-1-yl-5,6,7,8-tetrahydro-naphthalen-2-yloxymethyl)-phenyl]-morpholine

Intermediate 16 (0.61 mmol), morpholine (0.79 mmol), BINAP (0.06 mmol), palladium acetate (0.06 mmol), and cesium carbonate (0.92 mmol) was dissolved in 3 mL of toluene and heated to 100° C. for 72 hr. The reaction was purified using silican gel chromatography. Product containing fractions were combined and concentrated under reduced pressure to give 4-[7-(4-Morpholin-4-yl-benzyloxy)-1,2,3,4-tetrahydro-naphthalen-1-yl]-piperazine-1-carboxylic acid tert-butyl ester. ¹³C NMR (CDCl₃, 100 MHz) 21.5, 22.2, 28.6, 29.1, 48.4, 49.5, 63.4, 67.1, 70.0, 79.6, 113.4, 114.1, 115.9, 128.8, 129.1, 129.9, 130.8, 139.2, 151.2, 157.4.

The above compound was dissolved in 3 mL of 2M HCl/ethylether and stirred overnight at room temperature. The reaction was concentrated, tritrated with hexanes and ethylether and dried to provide the title compound.

### EXAMPLE 82

### 1-[7-(4-Piperidin-1-yl-benzyloxy)-1,2,3,4-tetrahydro-naphthalen-1-yl]-piperazine

Intermediate 16 was reacted with piperidine following the procedure detailed for Example 83, preparation 19 to afford 4-[7-(4-piperidin-1-yl-benzyloxy)-1,2,3,4-tetrahydro-naphthalen-1-yl]-piperazine-1-carboxylic acid tert-butyl ester; MS 506.4 [M+H]. The above compound was dissolved in 1 mL of 2M HCl/ether and stirred at rt for 8 h. The reaction was concentrated and dried to afford the title compound as a white solid.

### Preparation 20

### EXAMPLE 83

### 1-[7-(3′,4′-Dichloro-biphenyl-4-ylmethoxy)-1,2,3,4-tetrahydro-naphthalen-1-yl-1-piperazine

Intermediate 16 (0.24 mmol), 3,4 dichlorophenyl boronic acid (0.29 mmol), tetrakistriphenylphospine (0.02 mmol), potassium phosphate (0.48 mmol) in 2 mL of dioxane is heated to 100° C. for 15 hr. The reaction was concentrated and purified on silican gel chromatography to afford 4-[7-(3′,4′-dichloro-biphenyl-4-ylmethoxy)-1,2,3,4-tetrahydro-naphthalen-1-yl]-piperazine-1-carboxylic acid tert-butyl ester; MS 567.2 [M+H].

The above compound was dissolved in 1 mL of 2M HCl/diethylether solution wih 0.5 mL of chloroform and stirred at rt for 1 h. the reaction is concentrated to afford the title compound as a white solid.

### EXAMPLE 84

### 4-{4-[8-(4-Methyl-piperazin-1-yl)-5,6,7,8-tetrahydro-naphthalen-2-yloxymethyl]-phenyl}-morpholine

To 4-[7-(4-morpholin-4-yl-benzyloxy)-1,2,3,4-tetrahydro-naphthalen-1-yl]-piperazine-1-carboxylic acid tert-butyl ester (77 mg), prepared above in Example 83, is added 2 mL of a 1.0 M solution of lithium aluminum hydride in tetrahydrofuran. The reaction is stirred at 60° C. for 5 h and quenched with 1N sodium hydroxide. The reaction is extracted with methylene chloride, concentrated, and purified by silica gel chromatography to afford the title compound.

### EXAMPLE 85

### 4-{3-[8-(4-Methyl-piperazin-1-yl)-5,6,7,8-tetrahydro-naphthalen-2-yl]-piperidine-1-carbonyl}-benzoic acid methyl ester

The title compound was synthesized in the manner detailed in preparation 14, using example 71 as starting material. Following purification by silica gel chromatography (8% MeOH/CH₂Cl₂ eluent) the product was isolated as an oil (48% yield). MS 476.2 [M+H].

### Preparation 21

### EXAMPLE 86

### [4-(1-Hydroxy-1-methyl-ethyl)-phenyl]-{3-[8-(4-methyl-piperazin-1-yl)-5,6,7,8-tetrahydro-naphthalen-2-yl]-piperidin-1-yl}-methanone

4-{3-[8-(4-Methyl-piperazin-1-yl)-5,6,7,8-tetrahydro-naphthalen-2-yl]-piperidine-1-carbonyl}-benzoic acid methyl ester (55 mg, 0.12 mmol) was dissolved in 1 ml THF and chilled in an ice bath. MeMgBr (3.0 M, 385 uL, 1.2 mmol) was added and the reaction mixture was warmed to room temperature and stirred for 18 h. After quenching by slow addition of aq. ammonium chloride solution, the reaction mixture was diluted with water and extracted with dichloromethane. The combined organics were dried (Na₂SO₄) and concentrated to afford crude product. Purification on a silica gel flash column (10% MeOH/CH₂Cl₂, eluent) afforded 19 mg of the title compound as a foam (34% yield). MS 476.2 [M+H].

### Preparation 22

### EXAMPLE 87

### {3-[8-(4-Methyl-piperazin-1-yl)-5,6,7,8-tetrahydro-naphthalen-2-yl]-piperidin-1-yl}-[4-(morpholine-4-carbonyl)-phenyl]-methanone

Morpholine (63 ul, 0.72 mmol) was dissolved in 2 ml dichloromethane. Trimethyl aluminium (2.0 M in toluene, 312 ul, 62 mmol) was added and the mixture was stirred for 20 min. 4-{3-[8-(4-Methyl-piperazin-1-yl)-5,6,7,8-tetrahydro-naphthalen-2-yl]-piperidine-1-carbonyl)benzoic acid methyl ester (55 mg, 0.12 mmol) in 1 ml dichloromethane was added and the resulting mixture was stirred for 18 h at 50° C. After cooling, the reaction was quenched by slow addition of methanol, followed by water. After dilution with dichloromethane, the mixture was filtered through celite and concentrated to an oil. Purification was accomplished on a silica gel flash column (eluent: 5% MeOH/0.5% NH₄OH/CH₂Cl₂) and afforded the title compound in 20% yield. MS 531.2 [M+H].

### Preparation 23

### EXAMPLE 88

### 8-(4-Methyl-piperazin-1-yl)-5,6,7,8-tetrahydro-naphthalene-2-carboxylic acid 3,4-dichloro-benzylamide

Trifluoro-methanesulfonic acid 8-(4-methyl-piperazin-1-yl)-5,6,7,8-tetrahydro-naphthalen-2-yl ester (100 mg, 0.26 mmol), (Ph₃P)₂Pd Cl₂ (37 mg, 0.053 mmol), triethylamine (288 ul, 2.08 mmol) and 3,4-dichlorobenzyl amine (104 ul, 0.78 mmol) were combined and heated at 90° C. for 18 h in a CO atmosphere. The mixture was then cooled, diluted with dichloromethane, filtered through celite and concentrated to an oil. Purification by silica gel chromatography (3-6% MeOH in CH₂Cl₂ eluent) afforded the title compound in 27% yield. ¹³CNMR (100 MHz, CDCl₃) 21.53, 22.0, 29.8, 31.1, 43.0, 45.0, 55.4, 62.7, 125.4, 126.9, 127.4, 129.7, 129.8, 130.8, 131.5, 131.9, 132.8, 139.3, 143.1, 167.8.

### EXAMPLE 89

### 8-(4-Methyl-piperazin-1-yl)-5,6,7,8-tetrahydro-naphthalene-2-carboxylic acid (3,4-dichloro-phenyl)-amide

The title compound was synthesized as detailed in Preparation 23 utilizing trifluoro-methanesulfonic acid 8-(4-methyl-piperazin-1-yl)-5,6,7,8-tetrahydro-naphthalen-2-yl ester and 3,4-dichloroaniline. The desired product was obtained in 12% yield. MS 418.2 [M+H]. ¹³CNMR (100 MHz, CDCl₃) 21.5, 21.9, 29.9, 45.0, 55.2, 62.6, 119.9, 122.2, 125.6, 127.2, 127.6, 129.8, 130.6, 132.1, 138.1, 143.6, 166.2.

### EXAMPLE 90

8-(4-Methyl-piperazin-1-yl)-5,6,7,8-tetrahydro-naphthalene-2-carboxylic acid 3,4-difluoro-benzylamide was synthesized as detailed in Preparation 23 utilizing 8-(4-methyl-piperazin-1-yl)-5,6,7,8-tetrahydro-naphthalene-2-carboxylic acid methyl ester (Intermediate 9) and 3,4-difluorobenzyl amine. The desired product was isolated in 38% yield. MS 400.3 [M+H]. Diagnostic ¹³C NMR (100 MHz, CDCl₃) 21.6, 21.7, 29.9, 43.1, 46.0, 55.8, 62.7, 116.7, 117.5, 123.8, 125.2, 126.8, 129.5, 131.7, 138.6, 142.9.

1. A compound of the formula 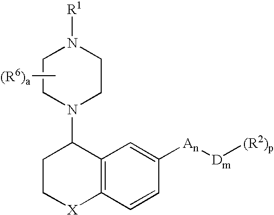
wherein
X is CH₂ or O;
n is zero or one;
m is zero or one;
p is zero or one;
R¹ is hydrogen, (C₁-C₆)alkyl, (C₁-C₄)alkyl-aryl wherein said aryl moiety is phenyl or naphthyl, wherein said aryl moiety may optionally be substituted with one or more substituents independently selected from (C₁-C₆)alkyl, (C₁-C₆)alkoxy, trifluoromethyl, cyano and halo;
A is absent or a group of the formula G¹, G², G^{2a}, G³, G⁴, G⁵, and G⁶ depicted below, 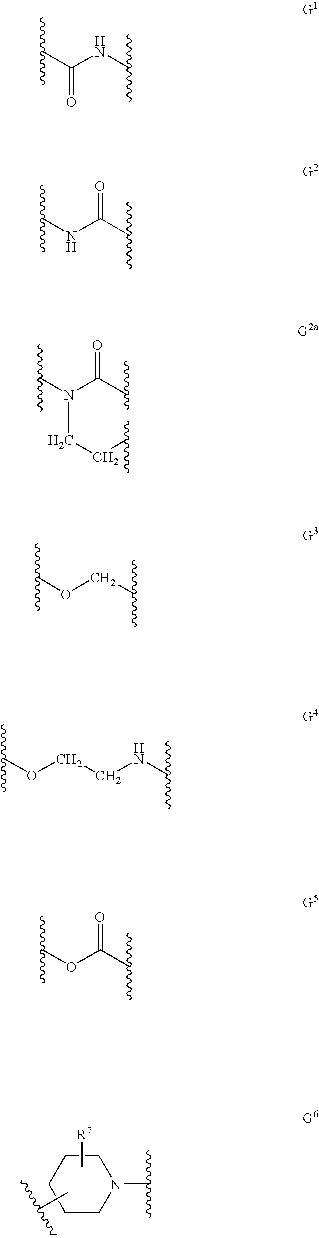
wherein the C═O and CH₂ of G^{2a} bind to two adjacent carbon atoms of the D moiety so that G^{2a} forms a six membered ring including two adjacent carbon atoms of the D moiety;
wherein if n is zero and m is one, the tetrahydronaphthyl moiety bonds to D, and if both n and m are zero and p is one the tetrahydronaphthyl moiety bonds to R², and if n is one, m is zero and p is one A bonds to R²;
D is a group of the formula depicted below, 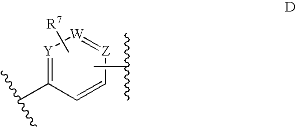
wherein W, Y and Z are independently C or N;
wherein R⁷ is H or optionally one to four substituents independently selected from chloro, fluoro, bromo, iodo, (C₁-C₈)alkyl, (C₁-C₈)perfluoroalkyl, wherein said alkyl or perfluoroalkyl is branched or linear, (C₁-C₈)hydroxyalkyl-, —CH₂NR⁸NR⁹, wherein R⁸ and R⁹ are independently H or (C₁-C₈)alkyl-, (C₁-C₈)alkoxy, (C₄-C₈)cycloalkyloxy, (C₄-C₈)cycloalkenyloxy, (C₁-C₈)alkoxy-(C₁-C₈)alkyl-, or R⁷ is a 5 to 7 membered non-aromatic heterocyclic ring having in addition to carbon atoms one to three heteroatoms independently selected from nitrogen, oxygen or sulfur atom or any combination thereof with the proviso that said ring cannot contain two adjacent oxygen atoms or two adjacent sulfur atoms; or,
R⁷ is —CONR⁴R⁵ wherein R⁴ and R⁵ are independently selected from (C₁-C₈)alkyl, (C₁-C₈)alkoxy benzyl, or R⁴ and R together with the nitrogen to which they are attached form a 5 to 7 membered heteroalkyl ring that may contain from zero to three heteroatoms selected from nitrogen, sulfur and oxygen in addition to the nitrogen of the —CONR⁴R⁵ group, wherein when any of said heteroatoms is nitrogen it may be optionally substituted with (C₁-C₈)alkyl or benzyl, with the proviso that said ring cannot contain two adjacent oxygen atoms or two adjacent sulfur atoms;
wherein R⁷ is H, (C₁-C₈)alkyl, aryl, heteroaryl, aryl(C₁-C₆)alkyl-, aryl(C₁-C₆)alkyl-O—, aryl-(C═O)—, heteroaryl(C₁-C₆)alkyl-, heteroaryl(C₁-C₆)O—, heteroaryl-(C═O)— wherein aryl is phenyl or naphthyl, and heteroaryl is a 5 to 7 membered aromatic ring containing from one to four heteroatoms in the ring selected from oxygen, nitrogen and sulfur, with the proviso that said ring cannot contain two adjacent oxygen atoms or two adjacent sulfur atoms and wherein the foregoing phenyl, naphthyl, and heteroaryl rings may be optionally substituted with one to three substituents independently selected from chloro, fluoro, bromo, iodo, (C₁-C₈)alkyl, (C₁-C₈)perfluoroalkyl, wherein said alkyl or perfluoroalkyl is branched or linear, (C₁-C₈)hydroxyalkyl-, (C₁-C₈)alkoxy, (C₁-C₈)alkoxy-(C₁-C₈)alkyl-, aryl, heteroaryl, aryl(C₁-C₆)alkyl-, aryl(C₁-C₆)alkyl-O—, aryl-(C═O)—, heteroaryl(C₁-C₆)alkyl-, hetereoaryl(C₁-C₆)O—, heteroaryl-(C═O)— wherein aryl is phenyl, naphthyl or 1,2,3,4-tetrahydro-naphthalenyl, and heteroaryl is a 5 to 7 membered aromatic ring containing from one to four heteroatoms in the ring selected from oxygen, nitrogen and sulfur, with the proviso that said ring cannot contain two adjacent oxygen atoms or two adjacent sulfur atoms;
R⁶ is (C₁-C₈)alkyl, wherein said alkyl is branched or linear; and,
a is 1 to 8;
or, a pharmaceutically acceptable salt thereof.

2. A compound according to claim 1 wherein R² is aryl, heteroaryl, aryl(C₁-C₆)alkyl-, aryl(C₁-C₆)alkyl-O—, aryl-(C═O)—, heteroaryl(C₁-C₆)alkyl-, hetereoaryl(C₁-C₆)O—, heteroaryl-(C═O)— wherein aryl is phenyl, naphthyl or 1,2,3,4-tetrahydro-naphthalenyl, and heteroaryl is selected from pyridyl, pyrrolyl, pyrimidyl, pyrazolyl, imidazolyl, triazolyl, tetrazolyl, oxazolyl, isoxazolyl, oxadiazolyl, thienyl, thiazolyl and isothiazoly and wherein the foregoing phenyl, naphthyl, 1,2,3,4-tetrahydro-naphthalenyl, and heteroaryl rings may be optionally substituted with one to three substituents independently selected from chloro, fluoro, bromo, iodo, (C₁-C₈)alkyl, (C₁-C₈)perfluoroalkyl, wherein said alkyl or perfluoroalkyl is branched or linear, (C₁-C₈)hydroxyalkyl-, (C₁-C₈)alkoxy, (C₁-C₈)alkoxy-(C₁-C₈)alkyl-, aryl, heteroaryl, aryl(C₁-C₆)alkyl-, aryl(C₁-C₆)alkyl-O—, aryl-(C═O)—, heteroaryl(C₁-C₆)alkyl-, hetereoaryl(C₁-C₆)O—, heteroaryl-(C═O)— wherein aryl is phenyl, naphthyl or 1,2,3,4-tetrahydro-naphthalenyl, and heteroaryl is selected from pyridyl, pyrrolyl, pyrimidyl, pyrazolyl, imidazolyl, triazolyl, tetrazolyl, oxazolyl, isoxazolyl, oxadiazolyl, thienyl, thiazolyl and isothiazoly.

3. A compound according to claim 1 wherein R⁷ is one to three substituents independently selected from the group consisting of phenyl, naphthyl, 1,2,3,4-tetrahydro-naphthalenyl, tetrahydropyranyl, morpholinyl, azetidinyl, pyrrolidinyl, piperidyl, piperazinyl, morpholinyl, thiomorpholinyl, hexahydroazepinyl, diazepinyl, oxazepinyl, thiazepinyl, oxadiazepinyl, thiadiazepinyl or triazepinyl, oxetanyl, tetrahydrofuranyl and wherein each said substituent may be independently substituted with from zero to three substituents independently selected from (C₁-C₈)alkyl chloro, fluoro, bromo, iodo, (C₁-C₈)alkyl, (C₁-C₈)perfluoroalkyl, wherein said alkyl or perfluoroalkyl is branched or linear, (C₁-C₈)hydroxyalkyl-, (C₁-C₈)alkoxy, (C₁-C₈)alkoxy-(C₁-C₈)alkyl-, aryl(C₁-C₆)alkyl.

4. A compound according to claim 1 wherein R⁷ is —CONR⁴R⁵ wherein R⁴ and R⁵ together with the nitrogen to which they are attached form a heteroalkyl ring selected from piperidine, N-(C₁-C₆)alkylpiperazine and morpholine.

5. A compound according to claim 1 wherein R¹ is selected from hydrogen, (C₁-C₆)alkyl, (C₁-C₄)alkyl-aryl wherein said aryl moiety is phenyl or naphthyl, and —C(═O)—O(C₁-C₈)alkyl.

6. A compound according to claim 1 wherein R¹ is selected from hydrogen, methyl, ethyl and benzyl.

7. A compound according to claim 1 wherein X is carbon.

8. A compound according to claim 1 wherein X is oxygen.

9. A compound according to claim 1 wherein one of W, Y or Z is nitrogen.

10. A compound according to claim 1 wherein n, m and p are one.

11. A compound according to claim 1 wherein n is zero, m is one and p is one.

12. A compound according to claim 1 wherein n is zero, m is zero and p is one.

13. A compound according to claim 1 wherein n is one, m is zero and p is one.

14. A compound according to claim 1 wherein n is one, m is one and p is zero.

15. A compound according to claim 1 selected from the group consisting of
N-{8-[(2-Dimethylamino-ethyl)ethyl-amino]-5,6,7,8-tetrahydro-naphthalen-2-yl}-4-trifluoromethyl-benzamide;
N-{8-[(2-Dimethylamino-ethylyethyl-amino]-5,6,7,8-tetrahydro-naphthalen-2-yl}-4-fluoro-benzamide;
4-tert-Butyl-N-{8-[(2-dimethylamino-ethyl)-ethyl-amino]-5,6,7,8-tetrahydro-naphthalen-2-yl}-benzamide;
1-[7-(4-Benzyl-phenyl)-1,2,3,4-tetrahydro-naphthalen-1-yl]-4-methyl-piperazine;
1-[7-(4-Benzyloxy-phenyl)-1,2,3,4-tetrahydro-naphthalen-1-yl]-4-methyl-piperazine;
1-Methyl-4-(7-phenyl-1,2,3,4-tetrahydro-naphthalen-1-yl)-piperazine;
1-[7-(4-Fluoro-phenyl)-1,2,3,4-tetrahydro-naphthalen-1-yl]-4-methyl-piperazine;
1-[7-(3,5-Dichloro-phenyl) 1,2,3,4-tetrahydro-naphthalen-1-yl]-4-methyl-piperazine;
1-[7-(2-Methoxy-phenyl)-1,2,3,4-tetrahydro-naphthalen-1-yl]-4-methyl-piperazine;
1-Methyl-4-[7-(4-trifluoromethyl-phenyl)-1,2,3,4-tetrahydro-naphthalen-1-yl]-piperazine;
1-[7-(3,4-Dimethoxy-phenyl)-1,2,3,4-tetrahydro-naphthalen-1-yl]-4-methyl-piperazine;
1-(7-Biphenyl-4-yl-1,2,3,4-tetrahydro-naphthalen-1-yl)-4-methyl-piperazine;
1-[7-(2,3-Dihydro-benzo[1,4]dioxin-6-yl)-1,2,3,4-tetrahydro-naphthalen-1-yl]-4-methyl-piperazine;
8-(4-Methyl-piperazin-1-yl)-5,6,7,8-tetrahydro-naphthalene-2-carboxylic acid [1-(4-methoxy-phenylyethyl]-amide;
8-(4-Methyl-piperazin-1-yl)-5,6,7,8-tetrahydro-naphthalene-2-carboxylic acid 4-tert-butyl-benzylamide;
8-(4-Methyl-piperazin-1-yl)-5,6,7,8-tetrahydro-naphthalene-2-carboxylic acid 4-trifluoromethyl-benzylamide;
8-(4-Methyl-piperazin-1-yl)-5,6,7,8-tetrahydro-naphthalene-2-carboxylic acid (1,2,3,4-tetrahydro-naphthalen-1-yl)-amide;
8-(4-Methyl-piperazin-1-yl)-5,6,7,8-tetrahydro-naphthalene-2-carboxylic acid [2-(3-trifluoromethyl-phenyl)ethyl]-amide;
8-(4-Methyl-piperazin-1-yl)-5,6,7,8-tetrahydro-naphthalene-2-carboxylic acid 4-chloro-benzylamide;
8-(4-Methyl-piperazin-1-yl)-5,6,7,8-tetrahydro-naphthalene-2-carboxylic acid 4-fluoro-benzylamide;
8-(4-Methyl-piperazin-1-yl)-5,6,7,8-tetrahydro-naphthalene-2-carboxylic acid (furan-2-ylmethyl)amide;
8-(4-Methyl-piperazin-1-yl)-5,6,7,8-tetrahydro-naphthalene-2-carboxylic acid 2-chloro-benzylamide;
8-(4-Methyl-piperazin-1-yl)-5,6,7,8-tetrahydro-naphthalene-2-carboxylic acid 3-trifluoromethyl-benzylamide;
8-(4-Methyl-piperazin-1-yl)-5,6,7,8-tetrahydro-naphthalene-2-carboxylic acid 3-fluoro-benzylamide;
8-(4-Methyl-piperazin-1-yl)-5,6,7,8-tetrahydro-naphthalene-2-carboxylic acid 2-methyl-benzylamide;
8-(4-Methyl-piperazin-1-yl)-5,6,7,8-tetrahydro-naphthalene-2-carboxylic acid 2-methoxy-benzylamide;
8-(4-Methyl-piperazin-1-yl)-5,6,7,8-tetrahydro-naphthalene-2-carboxylic acid 3-methoxy-benzylamide;
8-(4-Methyl-piperazin-1-yl)-5,6,7,8-tetrahydro-naphthalene-2-carboxylic acid (2,5-dimethyl-2H-pyrazol-3-yl)-amide;
8-(4-Methyl-piperazin-1-yl)-5,6,7,8-tetrahydro-naphthalene-2-carboxylic acid (5-tert-butyl-2-methyl-2H-pyrazol-3-yl-amide;
8-(4-Methyl-piperazin-1-yl)-5,6,7,8-tetrahydro-naphthalene-2-carboxylic acid [1-(4-chloro-phenyl)ethyl]-amide;
8-(4-Methyl-piperazin-1-yl)-5,6,7,8-tetrahydro-naphthalene-2-carboxylic acid 3-chloro-benzylamide;
8-(4-Methyl-piperazin-1-yl)-5,6,7,8-tetrahydro-naphthalene-2-carboxylic acid (pyridin-2-ylmethyl)-amide;
8-(4-Methyl-piperazin-1-yl)-5,6,7,8-tetrahydro-naphthalene-2-carboxylic acid (4-chloro-phenyl)amide;
8-(4-Methyl-piperazin-1-yl)-5,6,7,8-tetrahydro-naphthalene-2-carboxylic acid (5-methyl-[1,3,4]thiadiazol-2-yl)-amide;
8-(4-Methyl-piperazin-1-yl)-5,6,7,8-tetrahydro-naphthalene-2-carboxylic acid 3,4-difluoro-benzylamide;
8-(4-Methyl-piperazin-1-yl)-5,6,7,8-tetrahydro-naphthalene-2-carboxylic acid 4-methoxy-benzylamide;
8-(4-Methyl-piperazin-1-yl)-5,6,7,8-tetrahydro-naphthalen-2-carboxylic acid pyridin-2-ylamide;
1-Methyl-4-(7-o-tolyl-1,2,3,4-tetrahydro-naphthalen-1-yl)-piperazine;
1-[7-(3,4-Dichloro-phenyl)-1,2,3,4-tetrahydro-naphthalen-1-yl]-4-methyl-piperazine;
1-[7-(3-Methoxy-phenyl)-1,2,3,4-tetrahydro-naphthalen-1-yl]-4-methyl-piperazine;
4-tert-Butyl-N-[8-(4-methyl-piperazin-1-yl)-5,6,7,8-tetrahydro-naphthalen-2-yl]-benzamide;
2-Methoxy-N-[8-(4-methyl-piperazin-1-yl)-5,6,7,8-tetrahydro-naphthalen-2-yl]-4-morpholin-4-yl-benzamide;
4-Isopropoxy-N-[8-(4-methyl-piperazin-1-yl)-5,6,7,8-tetrahydro-naphthalen-2-yl]-benzamide;
4-Benzyloxy-N-[8-(4-methyl-piperazin-1-yl)-5,6,7,8-tetrahydro-naphthalen-2-yl]-benzamide;
Benzo[1,3]dioxole-5-carboxylic acid [8-(4-methyl-piperazin-1-yl)-5,6,7,8-tetrahydro-naphthalen-2-yl]-amide;
4-(Cyclohex-1-enyloxy)-N-[8-(4-methyl-piperazin-1-yl)-5,6,7,8-tetrahydro-naphthalen-2-yl]-benzamide;
N-(8-[(2-Dimethylamino-ethylymethyl-amino]-5,6,7,8-tetrahydro-naphthalen-2-yl)₂-fluoro-4-morpholin-4-yl-benzamide;
N-[8-(4-Methyl-piperazin-1-yl)-5,6,7,8-tetrahydro-naphthalen-2-yl]-4-trifluoromethoxy-benzamide;
1-Methyl-4-(7-pyridin-4-yl-1,2,3,4-tetrahydro-naphthalen-1-yl)piperazine;
1-Methyl-4-[7-(4-methyl-pyridin-3-yl)-1,2,3,4-tetrahydro-naphthalen-1-yl]-piperazine;
1-Methyl-4-[7-(6-methyl-pyridin-3-yl) 1,2,3,4-tetrahydro-naphthalen-1-yl)-piperazine; 1-[7-(6-Methoxy-pyridin-3-yl)-1,2,3,4-tetrahydro-naphthalen-1-yl]-4-methyl-piperazine;
1-Methyl-4-(7-pyridin-2-yl-1,2,3,4-tetrahydro-naphthalen-1-yl)piperazine;
4-{5-[8-(4-Methyl-piperazin-1-yl)-5,6,7,8-tetrahydro-naphthalen-2-yl]-pyridin-2-yl}morpholine;
(+)-1-Methyl-4-[7-(4-methyl-pyridin-3-yl)-1,2,3,4-tetrahydro-naphthalen-1-yl]-piperazine;
(−)-1-Methyl-4-[7-(4-methyl-pyridin-3-yl)-1,2,3,4-tetrahydro-naphthalen-1-yl]-piperazine;
1-Methyl-4-[6-(4-methyl-pyridin-3-yl)-chroman-4-yl]-piperazine;
1-Methyl-4-(6-pyridin-4-yl-chroman-4-yl)-piperazine;
4-{5-[4-(4-Methyl-piperazin-1-yl)-chroman-6-yl]-pyridin-3-yl}-morpholine;
1-[4-(4-Methyl-piperazin-1-yl)-chroman-6-yl]-1H-pyrrolo[2,3-b]pyridine;
1-Methyl-4-[6-(4-methyl-pyridin-3-yl)-chroman-4-yl]-piperazine;
1-Methyl-4-[7-(4-trifluoromethyl-benzyloxy)-1,2,3,4-tetrahydro-naphthalen-1-yl]-piperazine;
1-[7-(4-tert-Butyl-benzyloxy)-1,2,3,4-tetrahydro-naphthalen-1-yl]-4-methyl-piperazine;
6-Morpholin-4-yl-nicotinic acid 8-(4-methyl-piperazin-1-yl)-5,6,7,8-tetrahydro-naphthalen-2-yl ester;
N-[8-(4-Methyl-piperazin-1-yl)-5,6,7,8-tetrahydro-naphthalen-2-yl]-6-morpholin-4-yl-nicotinamide;
{4-[8-(4-Methyl-piperazin-1-yl]5,6,7,8-tetrahydro-naphthalen-2-ylcarbamoyl]-benzyl}-carbamic acid tert-butyl ester,
N-[8-(4-Methyl-piperazin-1-yl)-5,6,7,8-tetrahydro-naphthalen-2-yl]-4-trifluoromethyl-benzamide;
N-[8-(4-Methyl-piperazin-1-yl]5,6,7,8-tetrahydro-naphthalen-2-yl]-4-trifluoromethyl-benzamide;
2-[8-(4-Methyl-piperazin-1-yl)-5,6,7,8-tetrahydro-naphthalen-2-yl]-6-morpholin-4-yl-3,4-dihydro-2H-isoquinolin-1-one;
1-Methyl-4-(7-piperidinyl-1,2,3,4-tetrahydro-naphthalen-1-yl)piperazine;
1-Methyl-4-(7-piperidin-3-yl-1,2,3,4-tetrahydro-naphthalen-1-yl)-piperazine;
1-Methyl-4-[7-(1-methyl-piperidin-4-yl)-1,2,3,4-tetrahydro-naphthalen-1-yl]-piperazine;
(5-Fluoro-pyrimidin-2-yl)-{2-[8-(4-methyl-piperazin-1-yl)-5,6,7,8-tetrahydro-naphthalen-2-yloxy]-ethyl}-amine.
N-{2-[8-(4-Methyl-piperazin-1-yl)-5,6,7,8-tetrahydro-naphthalen-2-yloxy]-ethyl}-4-trifluoromethyl-benzamide;
N-{2-[8-(4-Methyl-piperazin-1-yl)-5,6,7,8-tetrahydro-naphthalen-2-yloxy]-ethyl}-4-trifluoromethyl-benzamide;
{4-[8-(4-Methyl-piperazin-1-yl-]5,6,7,8-tetrahydro-naphthalen-2-yl]-piperidin-1-yl}-(4-trifluoromethyl-phenyl)-methanone;
(3-[8-(4-Methyl-piperazin-1-yl)-5,6,7,8-tetrahydro-naphthalen-2-yl]-piperidin-1-yl}-(4-trifluoromethyl-phenyl)-methanone;
4-Aminomethyl-N-[8-(4-methyl-piperazin-1-yl)-5,6,7,8-tetrahydro-naphthalen-2-yl]-benzamide;
(+)-1-Methyl-4-(7-pyridin-4-yl-1,2,3,4-tetrahydro-naphthalen-1-yl)-piperazine;
(−)-1-Methyl-4-(7-pyridin-4-yl-1,2,3,4-tetrahydro-naphthalen-1-yl)-piperazine;
4-(1-Hydroxy-1-methyl-ethyl)-N-[8-(4-methyl-piperazin-1-yl)-5,6,7,8-tetrahydro-naphthalen-2-yl]-benzamide;
3-[8-(4-Methyl-piperazin-1-yl)-5,6,7,8-tetrahydro-naphthalen-2-yl]-6′-morpholin-4-yl-3,4,5,6-tetrahydro-2H-[1,2′]bipyridinyl;
(+)- and (−)-enantiomers thereof; and, pharmaceutically acceptable salts thereof.

16. A pharmaceutical composition comprising a compound according to claim 1, or a pharmaceutically acceptable salt thereof, and a pharmaceutically acceptable carrier.

17. A method of treating a disorder or condition in a mammal selected from depression, anxiety, depression with concomitant anxiety, post traumatic stress disorder, panic phobias, obsessive compulsive disorder (OCD), borderline personality disorder, sleep disorder, psychosis, seizures, dyskinesis, symptoms of Huntington's or Parkinson's diseases, spasticity, suppression of seizures resulting from epilepsy, cerebral ischemia, anorexia, faintness attacks, hypokinesia, cranial traumas, chemical dependencies, premature ejaculation, premenstrual syndrome (PMS) associated mood and appetite disorder, inflammatory bowel disease, modification of feeding behavior, blocking carbohydrate cravings, late luteal phase dysphoric disorder, tobacco withdrawal-associated symptoms, panic disorder, bipolar disorder, sleep disorders, jet lag, cognitive dysfunction, hypertension, bulimia, anorexia, obesity, cardiac arrhythmias, chemical dependencies and addictions selected from dependencies on, or addictions to nicotine or tobacco products, alcohol, benzodiazepines, barbiturates, opioids or cocaine; headache, stroke, traumatic brain injury (TBI), psychosis, Huntington's Chorea, tardive dyskinesia, hyperkinesia, dyslexia, schizophrenia, multi-infarct dementia, epilepsy, senile dementia of the Alzheimer's type (AD), Parkinson's disease (PD), attention deficit hyperactivity disorder (ADHD) and Tourette's Syndrome, comprising administering to a mammal in need of such treatment an amount of a compound according to claim 1, or a pharmaceutically acceptable salt thereof, that is effective in treating such disorder or condition.

18. A method of treating a disorder or condition in a mammal selected from depression, anxiety, depression with concomitant anxiety, post traumatic stress disorder, panic phobias, obsessive compulsive disorder (OCD), borderline personality disorder, sleep disorder, psychosis, seizures, dyskinesis, symptoms of Huntington's or Parkinson's diseases, spasticity, suppression of seizures resulting from epilepsy, cerebral ischemia, anorexia, faintness attacks, hypokinesia, cranial traumas, chemical dependencies, premature ejaculation, premenstrual syndrome (PMS) associated mood and appetite disorder, inflammatory bowel disease, modification of feeding behavior, blocking carbohydrate cravings, late luteal phase dysphoric disorder, tobacco withdrawal-associated symptoms, panic disorder, bipolar disorder, sleep disorders, jet lag, cognitive dysfunction, hypertension, bulimia, anorexia, obesity, cardiac arrhythmias, chemical dependencies and addictions selected from dependencies on, or addictions to nicotine or tobacco products, alcohol, benzodiazepines, barbiturates, opioids or cocaine; headache, stroke, traumatic brain injury (TBI), psychosis, Huntington's Chorea, tardive dyskinesia, hyperkinesia, dyslexia, schizophrenia, multi-infarct dementia, epilepsy, senile dementia of the Alzheimer's type (AD), Parkinson's disease (PD), attention deficit hyperactivity disorder (ADHD) and Tourette's Syndrome, comprising administering to a mammal in need of such treatment an amount of a compound according to claim 1 that is an effective antagonist, inverse agonist or partial agonist of 5-HT_{1A} or 5-HT_{1B} receptors or a combination of 5-HT_{1A} and 5-HT_{1B} receptors.
